(19) United States
(12) Patent Application Publication
(10) Pub. No.: US 2014/0378531 A1
(43) Pub. Date: Dec. 25, 2014

(54) INHIBITION OF PATTERN RECOGNITION RECEPTORS IN PANCREATIC CANCER TREATMENT USING TLR INHIBITORS

(71) Applicant: NEW YORK UNIVERSITY, New York, NY (US)

(72) Inventors: George Miller, Englewood, NJ (US); Atsuo Ochi, Long Island City, NY (US)

(73) Assignee: NEW YORK UNIVERSITY, New York, NY (US)

(21) Appl. No.: 14/376,269

(22) PCT Filed: Feb. 1, 2013
(86) PCT No.: PCT/US2013/024260

§ 371 Date: Aug. 1, 2014

Related U.S. Application Data
(60) Provisional application No. 61/593,412, filed on Feb. 1, 2012.

(51) Int. Cl. C12N 15/113; A61K 31/7088; G01N 33/574; A61K 45/06

(52) U.S. Cl. CPC C12N 15/1138; A61K 45/06; A61K 31/7088; G01N 33/57484; G01N 33/57438; C12N 2310/14; C12N 2320/30; C12N 2320/31; G01N 2500/02; G01N 2333/705
USPC 514/44 A; 536/24.5; 436/501; 435/7.1

(57) ABSTRACT

The disclosure herein relates to the novel finding that pattern recognition receptor activation is central to pancreatic cancer progression, and provides antagonists of pattern recognition receptors (PRRs), including the TLRs 4, 7, and 9, and the CLR dectin-1, for treatment and prevention of pancreatic cancer and pancreatic inflammation. The inventors have discovered that cancer development and progression can be prevented by pattern recognition receptor inhibition, a powerful finding with important clinical and therapeutic implications.

### CROSS REFERENCE TO RELATED APPLICATION

The present application is a PCT application of U.S. Provisional Application Ser. No. 61/593,412, filed Feb. 1, 2012, the entire contents of which are incorporated herein by reference.

### BACKGROUND OF THE DISCLOSURE

Pancreatic ductal adenocarcinoma is the 4^{th} most common cause of cancer death in the United States and is lethal in more than 95% of cases (Kuhn et al., 2010). Unlike most adenocarcinomas, pancreatic cancer is overwhelmingly comprised of stromal and desmoplastic elements, interspersed with islands of neoplastic epithelium (Korc, 2007; Miyamoto et al., 2004). Recent evidence suggests that—far from being a passive observer—pancreatic tumor stroma directly impacts cancer progression and clinical outcome. By releasing nutrient growth factors including insulin-like growth factor and platelet-derived growth factor into the tumor microenvironment, the stromal component of pancreatic cancer has been linked to tumor growth and invasiveness (De Wever and Mareel, 2003; Ding et al., 2005; Kleeff et al., 1998). Further, chemotherapy resistance has been correlated with the extent of tumor desmoplasia, as the stroma is thought to be a physical barrier preventing cytotoxic agents from reaching neoplastic epithelial cells (Hwang et al., 2008a). However, the activators of the tumor stroma and the precise interplay between the stroma and transformed ductal epithelial cells are poorly understood (Neesse et al., 2011).

Pattern recognition receptors—the most well-described being the Toll-like receptors (TLRs)—are protein receptors expressed on innate immune cells as well as on selected neoplastic tissues (Huang et al., 2008). The first-identified ligands of TLRs were conserved bacterial or viral molecular motifs denoted pathogen-associated molecular patterns (PAMPs). One ligand for TLRs is single stranded RNA, a common feature of viral genomes. However, TLRs can also be bound by byproducts of inflammation or cellular injury, denoted damage-associated molecular patterns (DAMPS). As such, TLRs provide powerful avenues linking innate immunity to environmental stimuli. TLRs transduce NF-κB and MAP Kinase signaling cascades, leading to cytokine production, further recruitment of inflammatory mediators, and intense inflammation (Aderem and Ulevitch, 2000; Wang et al., 2008). Other pattern recognition receptors include C-type lectin receptors (CLRs), such as dectin-1, which recognizes glycan epitopes present on a range of pathogens, including viruses, bacteria, and fungi.

A handful of recent investigations have found ligation of selected TLRs to have either pro-tumorigenic or anti-neoplastic effects, varying with the cancer subtype and experimental context (Huang et al., 2008). These dichotomous patterns are not necessarily contradictory, as the effects of selective TLR activation in cancer progression can be contingent on the particular role played by immunity and inflammation in a particular malignancy. Specifically, in neoplastic processes without an evident primary inflammatory component, TLR ligation has been found to break self-antigen tolerance, promoting anti-tumor immune responses.

Protective effects of TLR ligation are reportedly mediated in part by TLR-activated dendritic cells which initiate antigen-restricted immunity (Lore et al., 2003; Salem, 2011). Similarly, TLR4 activation has been found to be an integral element of tumor regression in adoptive transfer of tumor-specific T cells in the treatment of metastatic melanoma (Paulos et al., 2007). In neu transgenic mice, a model mimicking human Her-2/neu(+) breast cancer, ligation of TLR7 elicits tumor regression (Lu et al., 2010). Notably, tumor protective effects of TLR ligation in selected cancers can be further amplified in the wake of cytotoxic chemotherapy or radiation therapy, resulting from the subsequent release of high levels of endogenous ligands and commensal microbial products (Roses et al., 2008; Wang et al., 2008). By contrast, in ulcerative colitis induced colon carcinoma and Helicobacter pylori-associated gastric cancer, diseases governed by a primary inflammatory component, recent investigations have revealed an essential link between the pro-inflammatory environment subsequent to activation of TLRs—particularly TLR4—and the ensuing malignant degeneration and metastasis (Fukata and Abreu, 2008; Fukata et al., 2007).

The effects of selective TLR ligation in modulating pancreatic cancer, a disease governed by a desmoplastic stroma, have not been previously studied.

TLR7 is expressed intracellularly within endosomal compartments in a variety of cell types, where its ligation triggers an intense inflammatory response (Doyle and O'Neill, 2006; Lund et al., 2004). TLR7 signaling is evident in chronic pancreatitis, a predisposing factor for pancreatic cancer (Gold and Cameron, 1993). However, a role for TLR7 or other TLRs in pancreatic cancer has not been established.

### BRIEF SUMMARY OF THE DISCLOSURE

The disclosure herein provides the novel finding that TLR activation is central to pancreatic cancer progression, and provides antagonists of TLR signaling for treatment and prevention of pancreatic cancer. The inventors have discovered that cancer progression can be halted by TLR inhibition, a powerful finding with important clinical and therapeutic implications.

Inhibition of TLR signaling by receptor inhibition, receptor deletion, and blockade of downstream signaling pathways, prevents pancreatic cancer progression. These results constitute a novel role for TLR signaling in pancreatic inflammation and malignancy. The dependency of these effects on inflammation mediated by TLR4, TLR7, TLR9, dectin-1, NF-κB and MAP kinase signaling, and CD4⁺ T cells provides multiple therapeutic targets.

### BRIEF DESCRIPTION OF THE FIGURES

FIG. 1. High expression of TLR7 in stromal and epithelial compartments in pancreatic carcinoma. (a) Representative paraffin embedded sections of pancreata from six months-old WT or p48Cre;Kras^{G12D} mice and (b) human pancreatic cancer specimens stained with mAbs directed against TLR7. (c) The total number of CD45⁺ stromal leukocytes in pancreata of six months-old WT or p48Cre;Kras^{G12D} mice was determined using flow cytometry. (d) Pancreatic leukocyte (CD45⁺) and (e) parenchymal (CD45⁻) cellular subsets from WT and p48Cre;Kras^{G12D} mice were analyzed by flow cytometry for expression of TLR7. The fraction of cells expressing TLR7 for each subset is indicated. Data are representative experiments were repeated three times using 4 mice per group (***p<0.001). Human data is representative of 19 human pancreatic carcinoma specimens analyzed.

FIG. 2. TLR7 ligation accelerates pancreatic tumorigenesis. (a) p48Cre;Kras^{G12D} mice were administered saline or TLR7 ligand (ssRNA40) for 3 weeks (n=5/group). Pancreata were harvested and weighed. (b) Representative gross images of excised pancreata are shown. (c) Paraffin embedded sections were examined by H&E and (d) both the fraction of various grades of PanTNs and foci of invasive cancer were quantified. (e) Gimori's Trichrome and (f) CD45 staining were performed to assess both stromal fibrosis and leukocytic infiltration (n=5/group; ***p<0.001).

FIG. 3. TLR7 activation induces epithelial cell proliferation and the development of somatic mutations. (a) p48Cre;Kras^{G12D} mice were administered saline or TLR7 ligand (ssRNA40) for 3 weeks. Pancreata were stained for Ki67, (b) mutated p53, (c) p16, and (d) CK19, an epithelial cell marker (n=5/group; ***p<0.001).

FIG. 4. Inhibition of TLR7 protects against pancreatic tumor progression. (a) p48Cre;Kras^{G12D} mice were administered saline, caerulein, or caerulein+an oligonucleotide inhibitor of TLR7. Pancreata were (a) weighed, (b) assessed by H&E, (c) Trichrome, and (d) CD45 staining. Results were quantified by assessing 10 HPFs per mouse (n=4/group; ***p<0.001).

FIG. 5. TLR7 ligation exacerbates pancreatic fibro-inflammation and TLR7 inhibition is protective. (a) Caerulein pancreatitis was induced in WT mice. Selected mice were additionally treated with either TLR7 ligand or inhibitor (n=5/group). Pancreata were stained using H&E, Trichrome, or using mAbs directed against CD45 and α-SMA. (b) The surface area occupied by acinar structures, (c) fibrotic surface area, and (d) CD45 leukocytic infiltrate were quantified by examining 10 HPFs per pancreas (***p<0.001).

FIG. 6. TLR7^{-/-} chimeric p48Cre;Kras^{G12D} mice are protected from accelerated pancreatic carcinogenesis. p48Cre;Kras^{G12D} mice were irradiated and made chimeric by bone marrow transfer from WT or TLR7^{-/-} mice. At seven weeks, mice were treated with either saline or two doses of caerulein to accelerate carcinogenesis. Three weeks later, mice were sacrificed and pancreata assessed by (a) both H&E and Ki67 staining (b) The fraction of metaplastic and dysplastic ducts were measured and (c, d) the number of foci of invasive cancer was quantified using CK19 immunohistochemistry (n=5/group; *p<0.05, ***p<0.001).

FIG. 7. TLR7 mediated pancreatic tumorigenesis requires intact NF-κB and MAP Kinase signaling and CD4⁺ T cells. (a), (b) p48Cre;Kras^{G12D} mice were administered saline or TLR7 ligand (ssRNA40) for 3 weeks. Selected mice were simultaneously treated with the NEMO binding domain inhibitor or PD98059 to block NF-κB and MAP Kinasc signaling, respectively (n=3-5/group). (c) p48Cre;Kras^{G12D} mice were administered saline or TLR7 ligand (ssRNA40) for 3 weeks. Selected mice were simultaneously depleted of CD4⁺ T cells (n=4/group).

FIG. 8. Elevated levels of TLR agonists in the pancreatic cancer micro-environment. (a) Pancreatic lysates from p48Cre;Kras^{G12D} or WT mice were tested for TLR7 ligand levels on HEK-Blue cells (n=4 group). (b) Pancreatic ductal fluid from patients with pancreatic carcinoma was harvested at the time of operative duct transaction and analyzed for HMGB-1 and S100A9 expression by Western blotting (20 μg protein loaded per well).

FIG. 9. High pancreatic expression of TLR7 and levels of TLR7 ligands in benign fibro-inflammatory pancreatic disease. (a) Representative paraffin embedded pancreatic sections stained for TLR7 from patients with pancreatitis who underwent pancresatic resection or (b) caerulein-treated mice. (c) The total number of CD3⁺CD4⁺ T cells, CD3⁺CD8⁺ T cells, CD3⁻CD19⁺ B cells, CD11c⁺MHCII⁺ dendritic cells, Gr1⁻CD11b⁺ macrophages, and Gr1⁺CD11b⁺ neutrophils staining positively for TLR7 per pancreas were calculated for both saline and caerulein treated pancreata using flow cytometry. Averages of 5 mice per group is shown (***p<0.001). (d) Representative dot plots of pancreatic single-cell suspensions co-stained for CD45 and TLR7 shows increased expression of TLR7 in both CD45⁺ leukocytes and CD45⁻ parenchymal cells in benign inflammatory pancreatic disease. (e) Sub-gating on CD34⁻CD45⁻ CD133⁺ epithelial cells and CD34⁻CD45⁻CD146⁺ endothelial cells revealed increased expression on TLR7 on ductal epithelial cells in pancreatitis. Median fluorescent indexes (MFI) are shown. (f) Human pancreatic duct fluid from patients with pancreatitis were tested on HEK-Blue cells for the presence of TLR7 agonists (n=2/group). (g) Pancreatic lysates from saline and caerulein treated mice were tested for the presence of HMGB-1.

FIG. 10. TLR7 activation induces endocrine organ destruction and augments inflammation in the injured pancreas. (a) Paraffin embedded sections of pancreata from mice treated with caerulein or caerulein+ssRNA40 were stained for Insulin, MPO, and B220. (b) Pancreatic islet cell area and (c) the extent of cellular infiltrate was calculated (n=5/group; ***p<0.001).

FIG. 11. Specificity of effects of TLR7 ligation in the pancreas (a) WT mice were treated for 3 weeks with caerulein alone, caerulein+E. coli RNA, or caerulein+Adenine analogue. (b) To test the specificity of the effects of TLR7 agonists, WT and TLR7^{-/-} mice were treated for 3 weeks with caerulein or caerulein+ssRNA40, respectively. (c) To determine the effects of TLR7 ligation in absence of baseline pancreatic inflammation, WT mice were treated with either saline, ssRNA40 alone, caerulein alone, or caerulein+ssRNA40. (d) Representative H&E stained sections of liver, kidney, lung, and small intestinal tissue from mice treated caerulein+ssRNA40 is shown (n=3-6 mice/group for each experiment).

FIG. 12. TLR7^{-/-} mice are protected from pancreatic injury. WT or TLR7^{-/-} mice (n=3/group) were treated for one week with L-arginine which induced mild pancreatic edema and inflammation in WT mice. Conversely, TLR7^{-/-} animals were protected.

FIG. 13. Pancreatic stromal expansion requires signaling mechanisms downstream of TLR7. (a-c) WT mice were treated with caerulein for 6 weeks to induce severe pancreatitis. Selected chorts were additionally treated with an MKK inhibitor, a p38 inhibitor, a JNK inhibitor, or an IκB inhibitor. Pancreata were stained using (a) H&E, (b) Trichrome, and (c) using an mAb directed against CD45. Results were quantified by examining 10 HPFs per pancreas (n=3 mice/group; *p<0.05, **p<0.01, ***p<0.001).

FIG. 14. TLR7 signaling in leukocytes, but not in parenchyma, regulates pancreatic fibro-inflammation. (a-c) WT mice were made chimeric using either WT bone marrow cells (WT chimeric [WT]) or bone marrow cells from TLR7^{-/-} mice (TLR7^{-/-} chimeric [WT]). Similarly, TLR7^{-/-} were made chimeric using WT hone marrow (WT chimeric [TLR7^{-/-}]). After 7 weeks, caerulein pancreatitis was induced in all cohorts. (b) The acinar and (c) fibrotic surface areas were calculated by examining 10 HPFs per pancreas (n=4 mice/group; ***p<0.001).

FIG. 15. TLR7 agonists exacerbate pancreatic inflammation via NF-κB and MAP Kinase dependent mechanisms. Mice were treated for 3 weeks with caerulein to induce pancreatitis. Selected mice were additionally treated with TLR7 ligand (ssRNA40) which exacerbated inflammation, fibrosis, and organ destruction. However, NF-↓B^{-/-} mice and animals treated with the MAP Kinase inhibitor, PD98059, were protected from effects of TLR7 ligation.

FIG. 16. Pancreatic fibro-inflammation mediated by TLR7 activation requires CD4⁺ T cells but is independent of CD8⁺ T cells and B cells. WT mice or mice deficient in CD4⁺ T cells, CD8⁺ T cells, or B cells were treated for 3 weeks with caerulein+TLR7 ligand (ssRNA40). Absence of CD4⁺ T cells protected animals from effects of TLR7 ligation (n=3/group).

### DETAILED DESCRIPTION OF THE DISCLOSURE

The disclosure herein relates to the novel finding that pattern recognition receptor activation is central to pancreatic cancer progression, and provides antagonists of pattern recognition receptors (PRRs), including the TLRs 4, 7, and 9, and the CLR dectin-1, for treatment and prevention of pancreatic cancer and pancreatic inflammation. The inventors have discovered that cancer development and progression can be prevented by pattern recognition receptor inhibition, a powerful finding with important clinical and therapeutic implications.

The inventors have discovered that TLR expression is markedly increased on both stromal and epithelial cells in pancreatic cancer, thus establishing TLRs as activators in the pancreatic cancer microenvironment. Correspondingly, TLR activation dramatically accelerates pancreatic carcinogenesis, while inhibition of TLR signaling at several strata—including receptor inhibition, receptor deletion, and blockade of downstream signaling pathways—prevents pancreatic cancer progression. TLR activation is sufficient to induce additional somatic mutations, including altered expression of p53 and p16, in Kras transformed ductal epithelial cells. Collectively, these results constitute a novel role for TLR signaling in pancreatic inflammation and malignancy. The dependency of these effects on inflammation mediated by TLR4, TLR7, TLR9, dectin-1, NF-κB and MAP kinase signaling, and CD4⁺ T cells provides multiple therapeutic targets.

This disclosure provides methods of treating or preventing pancreatic cancer in a subject. In one embodiment, the method comprises administering an antagonist of at least one pattern recognition receptor chosen from TLR4, TLR7, TLR9, and dectin-1, in an amount sufficient to treat or prevent pancreatic cancer in said subject.

In another embodiment, the antagonist of at least one pattern recognition receptor chosen from TLR4, TLR7, TLR9, and dectin-1 may be administered in combination with one or more additional anti-cancer treatments. The one or more additional anti-cancer treatments can be surgery, radiation therapy, chemotherapy, and/or biological therapy.

This disclosure further provides methods of treating or preventing pancreatic inflammation in a subject, comprising administering an antagonist of at least one pattern recognition receptor chosen from TLR4, TLR7, TLR9, and dectin-1, in an amount sufficient to treat or prevent pancreatic inflammation in said subject.

### DEFINITIONS

As used herein, the terms “subject” and “patient” are used interchangeably and refer to an animal, preferably a mammal such as a non-primate (e.g., cows, pigs, horses, cats, dogs, rats etc.) or a primate (e.g., monkey and human), and most preferably a human.

As used herein, “treat” or “treating” refers to clinical intervention in an attempt to alter the disease course of the individual or cell being treated. Therapeutic effects of treatment include without limitation, alleviation of symptoms, diminishment of any direct or indirect pathological consequences of the disease, decreasing the rate of disease progression, amelioration or palliation of the disease state, and remission or improved prognosis. For example, treatment of a cancer patient may be reduction of tumor size, elimination of malignant cells, prevention of metastasis, or the prevention of relapse in a patient whose tumor has regressed.

The term “prevent” or “preventing” as used herein refers to minimizing, reducing or suppressing the risk of developing a disease state or parameters relating to the disease state or progression or other abnormal or deleterious conditions. Preventing a disease state includes without limitation, preventing occurrence or recurrence of disease, prevention of metastasis, or the prevention of relapse in a patient whose tumor has regressed.

As used herein, the terms “therapeutically effective amount” and “effective amount” are used interchangeably to refer to an amount of a composition of the invention that is sufficient to result in the prevention of the development, recurrence, or onset of cancer or inflammation and one or more symptoms thereof, to enhance or improve the prophylactic effect(s) of another therapy, reduce the severity and duration of cancer or inflammation, ameliorate one or more symptoms of cancer or inflammation, prevent the advancement of cancer, cause regression of cancer or inflammation, and/or enhance or improve the therapeutic effect(s) of additional anti-cancer or anti-inflammatory treatment(s).

A therapeutically effective amount can be administered to a patient in one or more doses sufficient to palliate, ameliorate, stabilize, reverse or slow the progression of the disease, or otherwise reduce the pathological consequences of the disease, or reduce the symptoms of the disease. The amelioration or reduction need not be permanent, but may be for a period of time ranging from at least one hour, at least one day, or at least one week or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition, as well as the route of administration, dosage form and regimen and the desired result.

As used herein, the term “antagonist” refers to a biological or chemical agent that acts within the body to reduce the physiological activity of another chemical or biological substance. In the present invention, the antagonist blocks, inhibits, reduces and/or decreases the activity of a Pattern Recognition Receptor (PRR) protein. In a preferred embodiment, the antagonist is an antagonist of TLR4, TLR7, TLR9, or dectin-1 signaling. The terms antagonist and inhibitor can be used interchangeably. Antagonists and inhibitors of PRRs reduce PRR activity by binding to PRRs, or can prevent or reduce PRR signaling by other mechanisms.

### Pattern Recognition Receptor Antagonists

Antagonists according to the invention can be peptides, polypeptides, proteins, antibodies, antisense oligonucleotides, RNAi/siRNA, ribozymes, small molecules, chemotherapeutic agents, and fragments, derivatives and analogs thereof. In a particular embodiment, the antagonist is a TLR7 antagonist. In another particular embodiment, a TLR7 antagonist is administered in combination with at least one of a TLR4 antagonist, a TLR9 antagonist, or a dectin-1 antagonist.

PRR antagonists and inhibitors are known in the art. For example, known TLR7/9 inhibitors include the TLR7/9 antagonist IRS954 developed by Dynavax (see for example Tomai et al., Drug Discovery Today, 2006) and 2′-β-methyl modified RNAs as described in Robbins et al., Molecular Therapy, 2007.

The present invention provides small molecule antagonists, including peptides and synthesised and naturally occurring organic and inorganic molecules that inhibit the pattern recognition receptors of the invention.

The terms “peptide”, “polypeptide”, and “protein”, as used herein, refer to a sequence of amino acid residues linked together by peptide bonds or modified peptide bonds. Typically, a polypeptide or protein is at least six amino acids long and a peptide is at least 3 amino acids long. The protein, polypeptide or peptide can be naturally occurring, recombinant, synthetic, or a combination of these. The protein, polypeptide or peptide can be a fragment of a naturally occurring protein or polypeptide. The terms polypeptide and peptide also encompass peptide analogues, peptide derivatives and peptidomimetic compounds. Such compounds are well known in the art and may have significant advantages over naturally occurring peptides, including, for example, greater chemical stability, increased resistance to proteolytic degradation, enhanced pharmacological properties (such as, half-life, absorption, potency and efficacy), altered specificity (for example, a broad-spectrum of biological activities) and/or reduced antigenicity.

The present invention contemplates the use of biologically inactive proteins or fragments of proteins that bind to and inhibit PRRs, particularly TLR4, TLR7, TLR9, or dectin-1. Examples include dominant negative mutants. Biologically inactive proteins or fragments contemplated by the present invention are those that have substantially less activity than the wild-type protein. Candidate inhibitory fragments can be selected from random fragments generated from the wild-type protein. Methods for generating the candidate polypeptide fragments are well known to workers skilled in the art. Biologically inactive proteins can also be generated, for example, by site-directed or random mutagenesis techniques of nucleic acids encoding the protein, or by inactivation of the protein by chemical or physical means.

In accordance with the present invention, there are provided antibodies that antagonize PRRs, particularly TLR4, TLR7, TLR9, or dectin-1. Such antibodies can be polyclonal or monoclonal and generated in any suitable species. Monoclonal antibodies may be native to the generating species or fully or partially humanized.

Various methods for the preparation of antibodies are known in the art (see, Antibodies: A Laboratory Manual, CSH Press, Eds., Harlow, and Lane (1988); Harlow, Antibodies, Cold Spring Harbor Press, NY (1989)). For example, antibodies can be prepared by immunizing a suitable mammalian host with a sample of whole cells isolated from a patient. Briefly, such methods of generating an immune response (e.g. humoral and/or cell-mediated) in a mammal, comprise the steps of: exposing the mammal's immune system to a PRR, such as TLR7, so that the mammal generates an immune response that is specific for PRR (e.g. generates antibodies that specifically recognize one or more PRR protein epitopes).

Antibodies can be produced by cell culture techniques, including the generation of monoclonal antibodies as described herein, or via transfection of antibody genes into suitable bacterial or mammalian cell hosts, in order to allow for the production of recombinant antibodies. In one technique, a PRR is initially injected into any of a wide variety of mammals (e.g., mice, rats, rabbits, sheep or goats). A superior immune response may be elicited if the sample is injected along with a carrier protein, such as bovine serum albumin or keyhole limpet hemocyanin. The sample is injected into the animal host, preferably according to a predetermined schedule incorporating one or more booster immunizations, and the animals are bled periodically so that titers of antibodies can be taken to determine adequacy of antibody formation. Polyclonal antibodies specific for the polypeptide may then be purified from such antisera by, for example, affinity chromatography using cells from the patient sample coupled to a suitable solid support.

A “monoclonal antibody” is an antibody obtained from a population of substantially homogeneous antibodies, i.e., the antibodies comprising the population are identical except for possible naturally occurring mutations that are present in minor amounts. Monoclonal antibodies specific for a PRR may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with a PRR). Such cell lines may be produced, for example, from spleen cells obtained from an animal immunized as described above. The spleen cells are then immortalized by, for example, fusion with a myeloma cell fusion partner, preferably one that is syngeneic with the immunized animal A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the PRR. Hybridomas having high reactivity and specificity for the PRR are important for therapeutic purposes. When the appropriate immortalized cell culture is identified, the cells can be expanded and antibodies produced.

In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction.

The antibodies of the invention can also be produced by recombinant means. Antibodies that bind specifically to a PRR can also be produced in the context of chimeric or complementarity-determining region grafted antibodies of multiple species origin. “Humanized” or human antibodies can also be produced, and are preferred for use in therapeutic contexts. Methods for humanizing murine and other non-human antibodies, by substituting one or more of the non-human antibody sequences for corresponding human antibody sequences, are well known [see for example, Jones et al., Nature 321: 522-525 (1986); Riechmann et al., Nature 332: 323-327 (1988); Verhoeyen et al., Science 239: 1534-1536 (1988), Carter et al., Proc. Natl. Acad. Sci. USA 89: 4285 (1993); and Sims et al., J. Immunol. 151: 2296 (1993)]. These humanized antibodies are designed to minimize unwanted immunological response toward rodent antihuman antibody molecules which limits the duration and effectiveness of therapeutic applications of those moieties in human recipients. Accordingly, preferred antibodies used in the therapeutic methods of the invention are those that are either fully human or humanized and that bind specifically to a PRR with high affinity but exhibit low or no antigenicity in the patient.

Fully human monoclonal antibodies of the invention can be generated using cloning technologies employing large human Ig gene combinatorial libraries (i.e., phage display) (Griffiths and Hoogenboom, Building an in vitro immune system: human antibodies from phage display libraries. In: Protein Engineering of Antibody Molecules for Prophylactic and Therapeutic Applications in Man, Clark, M. (Ed.), Nottingham Academic, pp 45-64 (1993); Burton and Barbas, Human Antibodies from combinatorial libraries. Id., pp 65-82). Fully human monoclonal antibodies of the invention can also be produced using transgenic mice engineered to contain human immunoglobulin gene loci (see also, Jakobovits, Exp. Opin. Invest. Drugs 7(4): 607-614 (1998); U.S. Pat. No. 6,162,963 issued 19 Dec. 2000; U.S. Pat. No. 6,150,584 issued 12 Nov. 2000; and U.S. Pat. No. 6,114,598 issued 5 Sep. 2000).

Anti-idiotypic antibodies are also contemplated in the invention. Anti-idiotypic antibodies of the invention can be used to induce an immune response to PRRs. The generation of anti-idiotypic antibodies is well known in the art; this methodology can readily be adapted to generate anti-idiotypic anti-protein of PRR antibodies that mimic a PRR protein epitope [see, for example, Wagner et al., Hybridoma 16:33-40 (1997); Foon et al., J. Clin. Invest. 96:334-342 (1995); Herlyn et al., Cancer Immunol. Immunother. 43:65-76 (1996)]. Anti-idiotypic antibodies can be used to further enhance cancer treatments as described herein.

Specificity of the PRR antibody or antibodies can be tested by many techniques known in the art. For example, the specificity may be determined by ELISA. PRR protein is used to coat the wells of a multi-well plate, using methods known in the art. Anti-PRR antibodies are added, and reactivity with the PRR is determined by antibody binding affinity. Other means of determining specificity, well known to those of skill in the art, include FACS analysis and immunochemistry.

This disclosure further provides antisense oligonucleotide inhibitors and antagonists of the PRRs TLR4, TLR7, TLR9, or dectin-1, and TLR4, TLR7, TLR9, or dectin-1 signaling, including but not limited to antisense oligonucleotides, RNAi, dsRNA, siRNA and ribozymes.

As used herein, “antisense oligonucleotide” refers to a stretch of single-stranded DNA or RNA, usually chemically modified, whose sequence (3′-5′) is complementary to the sense sequence of a molecule of mRNA. Antisense molecules thereby effectively inhibit gene expression by forming RNA/DNA duplexes, and offer a more targeted option for cancer therapy than chemotherapy or radiation. Antisense is believed to work by a variety of mechanisms, including physically blocking the ability of ribosomes to move along the messenger RNA, and hastening the rate at which the mRNA is degraded within the cytosol.

In order to avoid digestion by DNAse, antisense oligonucleotides are often chemically modified. For example, phosphorothioate oligodeoxynucleotides are stabilized to resist nuclease digestion by substituting one of the non-bridging phosphoryl oxygen of DNA with a sulfur moiety. Increased antisense oligonucleotide stability can also be achieved using molecules with 2-methoxyethyl (MOE) substituted backbones as described generally in U.S. Pat. No. 6,451,991, incorporated by reference, and US Published patent application US-2003-0158143-A1. Thus, in the combination and method of the invention, the antisense oligonucleotide is modified to enhance in vivo stability relative to an unmodified oligonucleotide of the same sequence. The modification may be a (2′-O-2-methoxyethyl) modification. The oligonucleotide may have a phosphorothioate backbone throughout, the sugar moieties of nucleotides 1-4 and 18-21 may bear 2′-O-methoxyethyl modifications and the remaining nucleotides may be 2′-deoxynucleotides.

The antisense oligonucleotide may be a 5-10-5 gap-mer methoxyl ethyl modified oligonucleotide corresponding to the sequence of a PRR. The antisense oligonucleotide may be from 10-25 bases in length, or from 15-23 bases in length, or from 18-22 bases in length, or 21 bases in length. In one embodiment, this oligonucleotide has a phosphorothioate backbone throughout.

It is understood in the art that an antisense oligonucleotide need not have 100% identity with the complement of its target sequence in order to be effective. The antisense oligonucleotides in accordance with the present invention, therefore, have a sequence that is at least about 70% identical to the complement of the target sequence. In one embodiment of the present invention, the antisense oligonucleotides have a sequence that is at least about 80% identical to the complement of the target sequence. In other embodiments, they have a sequence that is at least about 90% identical or at least about 95% identical to the complement of the target sequence, allowing for gaps or mismatches of several bases. Identity can be determined, for example, by using the BLASTN program of the University of Wisconsin Computer Group (GCG) software.

The antisense oligonucleotides according to the present invention are typically between 7 and 100 nucleotides in length. In one embodiment, the antisense oligonucleotides comprise from about 7 to about 50 nucleotides, or nucleotide analogues. In another embodiment, the antisense oligonucleotides comprise from about 7 to about 35 nucleotides, or nucleotide analogues. In other embodiments, the antisense oligonucleotides comprise from about 12 to about 35 nucleotides, or nucleotide analogues, and from about 15 to about 25 nucleotides, or nucleotide analogues.

In order for the antisense oligonucleotides of the present invention to function in inhibiting a PRR, it is necessary that they demonstrate adequate specificity for the target sequence and do not bind to other nucleic acid sequences in the cell. Therefore, in addition to possessing an appropriate level of sequence identity to the complement of the target sequence, the antisense oligonucleotides of the present invention should not closely resemble other known sequences. The antisense oligonucleotides of the present invention, therefore, should be less than 50% identical to any other mammalian nucleic acid sequence.

Reduction in the amount of a PRR may also be achieved using RNA interference or “RNAi”. RNAi or double-stranded RNA (dsRNA) directs gene-specific, post-transcriptional silencing in many organisms, including vertebrates. RNA interference mediated by siRNAs is known in the art to play an important role in post-transcriptional gene silencing (Zamore, Nature Struc. Biol., 8:746-750, 2001). In nature, siRNA molecules are typically 21-22 base pairs in length and are generated when long double-stranded RNA molecules are cleaved by the action of an endogenous ribonuclease. RNAi may be effected via directly introducing into the cell, or generating within the cell by introducing into the cell a suitable precursor (e.g. vector, etc.) of such an siRNA or siRNA-like molecule. An siRNA may then associate with other intracellular components to form an RNA-induced silencing complex (RISC). Transfection of mammalian cells with synthetic siRNA molecules having a sequence identical to a portion of a target gene leads to a reduction in the mRNA levels of the target gene (Elbashir et al., Nature, 411:4914498, 2001).

The oligonucleotide inhibitors according to the present invention can be siRNA molecules that are targeted to a gene of interest such that the sequence of the siRNA corresponds to a portion of said gene. RNA molecules used in the present invention generally comprise an RNA portion and some additional portion, for example a deoxyribonucleotide portion. The total number of nucleotides in the RNA molecule is suitably less than 49 in order to be effective mediators of RNAi. In preferred RNA molecules, the number of nucleotides is 16 to 29, more preferably 18 to 23, and most preferably 21-23. In certain embodiments of the invention, the siRNA or siRNA-like molecule is less than about 30 nucleotides in length. In a further embodiment, the siRNA or siRNA-like molecules are about 21-23 nucleotides in length. In an embodiment, siRNA or siRNA-like molecules comprise and 19-21 bp duplex portion, each strand having a 2 nucleotide 3′ overhang. In certain embodiments, the siRNA or siRNA-like molecule is substantially identical to a TLR7-encoding nucleic acid or a fragment thereof.

The double-stranded siRNA molecules can further comprise poly-T or poly-U overhangs at the 3′ and 5′ ends to minimise RNase-mediated degradation of the molecules. Typically, the overhangs at the 3′ and 5′ ends comprise two thymidine or two uridine residues. Design and construction of siRNA molecules is known in the art (see, for example, Elbashir, et al, Nature, 411:494498, 2001; Bitko and Batik, BMC Microbiol., 1:34, 2001). In addition, kits that provide a rapid and efficient means of constructing siRNA molecules by in vitro transcription are also commercially available (Ambion, Austin, Tex.; New England Biolabs, Beverly, Mass.) and may be used to construct the siRNA molecules of to the present invention.

The present invention further contemplates ribozyme oligonucleotide modulators that specifically target mRNA encoding a protein of interest. Ribozymes are RNA molecules having an enzymatic activity that enables the ribozyme to repeatedly cleave other separate RNA molecules in a nucleotide-sequence specific manner. Such enzymatic RNA molecules can be targeted to virtually any mRNA transcript, and efficient cleavage can be achieved in vitro (Kim et al., Proc. Natl. Acad. Sci. USA, 84:8788, 1987; Haseloff and Gerlach, Nature, 334:585, 1988; Cech, JAMA, 260:3030, 1988; Jefferies et al., Nucleic Acids Res., 17:1371, 1989).

Typically, a ribozyme comprises two portions held in close proximity: an mRNA binding portion having a sequence complementary to the target mRNA sequence, and a catalytic portion which acts to cleave the target mRNA. A ribozyme acts by first recognising and binding a target mRNA by complementary base-pairing through the target mRNA binding portion of the ribozyme. Once it is specifically bound to its target, the ribozyme catalyses cleavage of the target mRNA. Such strategic cleavage destroys the ability of a target mRNA to direct synthesis of an encoded protein. Having bound and cleaved its mRNA target, the ribozyme is released and can repeatedly bind and cleave new target mRNA molecules.

### Pharmaceutical Compositions and Administration

This disclosure provides pharmaceutical compositions comprising a PRR antagonist of the invention in combination with a biologically-acceptable carrier. For example, a PRR antagonist can be incorporated into pharmaceutical compositions suitable for administration. As used herein, “biologically acceptable carrier” is intended to include any and all solvents, dispersion media, coatings, antibacterial, and antifungal agents, isotonic and absorption delaying agents, and the like, compatible with biologics administration. Suitable carriers are described in the most recent edition of Remington's Pharmaceutical Sciences, a standard reference text in the field, which is incorporated herein by reference. Preferred examples of such carriers or diluents include, but are not limited to, water; saline; dextrose solution; human serum albumin; HBSS and other buffered solutions (including those with and without Ca⁺⁺ and Mg⁺⁺) known to those skilled in the relevant arts; and basal media. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A preferred oral dosage form, such as tablets or capsules, will contain the PRR inhibitor in an amount of from about 0.1 to about 500 mg, preferably from about 125 to about 200 mg, and more preferably from about 25 to about 150 mg. For parenteral administration (which is preferred), the TLR7 inhibitor will be employed in an amount within the range of from about 0.005 mg/kg to about 10 mg/kg and preferably from about 0.01 mg/kg to about 1 mg/kg.

### Combination Therapies for Treatment of Pancreatic Cancer

A composition of the invention may comprise one or more PRR antagonists in combination, including any combination of antibody inhibitors, oligonucleotide inhibitors, or small molecule inhibitors, as provided herein.

The pharmacologic compositions of the invention are further provided in combination with other therapeutic treatments to treat or prevent pancreatic cancer. The prophylactically and/or therapeutically effective amount or regimen of a PRR inhibitor can be administered herein in combination with one or more additional therapies.

Therefore, this disclosure provides a method of treating or preventing pancreatic cancer in a subject, comprising administering a therapeutically effective amount of an antagonist of at least one pattern recognition receptor chosen from TLR4, TLR7, TLR9, and dectin-1, and further comprising administering one or more additional anti-cancer treatments to said subject. In a particular embodiment, the one or more additional anti-cancer treatments is selected from the group consisting of surgery, radiation therapy, chemotherapy, and biological therapy.

The PRR antagonist and the one or more additional anti-cancer treatments can be administered separately, simultaneously, or sequentially, or in any manner best suited for tolerance by the patient. A combination of therapeutic agents may be administered to a subject by the same or different routes of administration. In alternative embodiments, two or more prophylactic or therapeutic agents are administered in a single composition.

A therapeutically effective amount or regimen of a composition of the invention can be administered to subjects that will, are or have undergone radiation therapy, chemotherapy, hormonal therapy and/or biological therapy including immunotherapy and/or targeted therapy, as well as those who have undergone surgery.

For example, a PRR antagonist can be administered in combination with one or more cancer therapeutic agents or anti-cancer agents. The terms “cancer therapeutic agent” and “anti-cancer agent” refer to any substance that inhibits or prevents the function, expression, or activity of cells and/or causes destruction of cells. The term is intended to include radioactive isotopes, chemotherapeutic agents, and toxins such as small molecule toxins or enzymatically active toxins of bacterial, fungal, plant or animal origin, including fragments and/or variants thereof. Examples of cytotoxic agents include, but are not limited to maytansinoids, yttrium, bismuth, ricin, ricin A-chain, doxorubicin, daunorubicin, taxol, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, sapaonaria officinalis inhibitor, and glucocorticoid and other chemotherapeutic agents.

The dosages of the one or more additional anti-cancer agents used in the combination therapy may be lower than those which have been or are currently being used to prevent, treat, and/or manage cancer in the patient. The recommended dosages of the one or more additional therapies currently used for the prevention, treatment, and/or management of cancer can be obtained from any reference in the art including, but not limited to, Hardman et al., eds., Goodman & Gilman's The Pharmacological Basis Of Therapeutics, 10th ed, Mc-Graw-Hill, N.Y., 2001; and Physician's Desk Reference (60^{th} ed., 2006), which are incorporated herein by reference in their entirety.

Thus, for example, a satisfactory result may be obtained employing the PRR antagonist in an amount for oral dosage within the range of from about 0.01 mg/kg to about 100 mg/kg and preferably from about 0.1 mg/kg to about 25 mg/kg in combination with the additional anti-cancer therapeutic agent in an amount within the range of from about 0.01 mg/kg to about 100 mg/kg and preferably from about 0.1 mg/kg to about 25 mg/kg with the PRR antagonist and the additional anti-cancer therapeutic agent being employed together in the same oral dosage form or in separate oral dosage forms taken at the same time.

In one form of treatment, a therapeutically effective amount or regimen of a composition of the invention is administered to a subject that is undergoing or has undergone surgery to remove a tumor, cancer cells or neoplasm. In a specific application, a therapeutically effective amount or regimen of a composition of the invention is administered to a subject concurrently or following surgery to remove a pancreatic tumor, cancer cells or neoplasm. In another specific application, a therapeutically effective amount or regimen of a composition of the invention is administered to a subject before surgery to remove a pancreatic tumor or neoplasm and can additionally be administered during and/or after surgery.

A therapeutically effective amount or regimen of a composition of the invention can be administered to patients with increased levels of the cytokine IL-6, which has been associated with the development of cancer cell resistance to different therapeutic regimens, such as chemotherapy and hormonal therapy.

### Combination Therapies for Treatment of Pancreatic Inflammation

A composition of the invention may comprise one or more PRR antagonists in combination, including any combination of antibody inhibitors, oligonucleotide inhibitors, or small molecule inhibitors, as provided herein.

The pharmacologic compositions of the invention are further provided in combination with other therapeutic treatments to treat or prevent pancreatic inflammation. The prophylactically and/or therapeutically effective amount or regimen of a PRR inhibitor can be administered herein in combination with one or more additional anti-inflammatory therapies.

Therefore, this disclosure provides a method of treating or preventing pancreatic inflammation in a subject, comprising administering a therapeutically effective amount of an antagonist of at least one pattern recognition receptor chosen from TLR4, TLR7, TLR9, and dectin-1, and further comprising administering one or more additional anti-inflammatory treatments to said subject.

Examples of anti-inflammatory compounds include non-steroidal anti-inflammatory drugs (NSAIDs) such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; steroids including, but not limited to, alclometasone diproprionate, amcinonide, beclomethasone dipropionate, betametasone, betamethasone benzoate, betamethasone diproprionate, betamethasone sodium phosphate, betamethasone valerate, clobetasol proprionate, clocortolone pivalate, hydrocortisone, hydrocortisone derivatives, desonide, desoximatasone, dexamethasone, flunisolide, flucoxinolide, flurandrenolide, halcinocide, medrysone, methylprednisolone, methprednisolone acetate, methylprednisolone sodium succinate, mometasone furoate, paramethasone acetate, prednisolone, prednisolone acetate, prednisolone sodium phosphate, prednisolone tebuatate, prednisone, triamcinolone, triamcinolone acetonide, triamcinolone diacetate, and triamcinolone hexacetonide; and other anti-inflammatory agents including, but not limited to, methotrexate, colchicine, allopurinol, probenecid, thalidomide or a derivative thereof, 5-aminosalicylic acid, retinoid, dithranol or calcipotriol, sulfinpyrazone and benzbromarone.

In a particular embodiment, the one or more additional anti-inflammatory treatments is selected from a non-steroidal anti-inflammatory drug (NSAID) or an anti-inflammatory steroid.

### Screening of Candidate Compounds

The invention further provides methods of screening candidate compounds, such as small molecule compounds, for usefulness in the treatment of pancreatic cancer or pancreatic inflammation. Also provided are methods of screening candidate compounds for activity as a PRR inhibitor.

Therefore, this disclosure provides methods of screening for an candidate agent for the treatment of pancreatic cancer, comprising contacting a pattern recognition receptor (PRR) with a test compound and detecting PRR activity in the presence of the test compound relative to PRR signaling in the absence of the test compound, wherein a decrease in PRR activity in the presence of the test compound relative to PRR activity in the absence of the test compound indicates that the test compound is a candidate agent for the treatment of pancreatic cancer. In a particular embodiment, the pattern recognition receptor is TLR4, TLR7, TLR9, or dectin-1. In another particular embodiment, the activity detected is signaling activity. Methods of detecting PRR activities, such as signaling activities, are known in the art.

Candidate compounds can be randomly selected or rationally selected or designed. As used herein, a candidate compound is said to be randomly selected when the compound is chosen randomly without considering the specific interactions involved in its potential association with molecular components of the stem cells, or other cells if culture is used. An example of random selection of candidate compounds is the use a chemical library or a peptide combinatorial library, or a growth broth of an organism. As used herein, a candidate-compound is said to be rationally selected or designed when the compound is chosen on a non-random basis which takes into account the sequence and or conformation of a target site or a process in connection with the compound's action. Candidate compounds can be rationally selected or rationally designed, for example, by using the nucleotide or peptide sequences that make up the target sites. For example, a rationally selected peptide can be a peptide whose amino acid sequence is identical to or a derivative of a functional consensus site of a PRR ligand.

The candidate compound may be isolated or unisolated, pure, partially purified, or in the form of a crude mixture, for example, it may be in the form of a cell, a lysate or extract derived from a cell, or a molecule derived from a cell. Where the candidate compound is present in a composition that comprises more than one molecular entity, it is contemplated that the composition may be tested as is and/or may optionally be fractionated by a suitable procedure and the fractionated sample tested using the method of the invention or another method to identify a particular fraction or component of the composition that acts as a PRR inhibitor. It is further contemplated that sub-fractions of test compositions may be re-fractionated and assayed repeatedly using the methods of the invention with the ultimate goal of excluding inactive components from the sub-combination identified as a PRR inhibitor. Intervening steps of compound isolation, purification and/or characterisation may be included as needed or appropriate.

Candidate compounds can be obtained in the form of large libraries of synthetic or natural compounds. Numerous means are currently used for random and directed synthesis of saccharide, peptide, and nucleic acid based compounds and are well-known in the art. Synthetic compound libraries are commercially available from a number of companies including Maybridge Chemical Co. (Trevillet, Cornwall, UK), Comgenex (Preton, N.J.), Brandon Associates (Merrimack, N.H.), and Microsource (New Milford, Conn.). A rare chemical library is available from Aldrich Milwaukee, Wis.). Combinatorial libraries are also available or can be prepared according to standard procedures. Alternatively, libraries of natural compounds in the form of bacterial, fungal, plant, and animal extracts are available from, for example, Pan Laboratories (Bothell, Wash.) or MycoSearch (North Carolina), or can be readily produced. Additionally, natural and synthetically produced libraries and compounds are readily modified through conventional chemical, physical, and biochemical means.

This disclosure further provides methods of determining susceptibility of a subject to the development of pancreatic cancer, comprising detecting the level of one or more PRRs in a sample of pancreatic tissue from said subject and comparing the level of said one or more PRRs in said subject to the level of the same one or more PRRs in a sample of pancreatic tissue from a control subject, where an increase in the level of one or more PRRs in the pancreatic tissue sample of said subject relative to the level of the same one or more PRRs in the pancreatic tissue sample of said control subject indicates that said subject has increased susceptibility to the development of pancreatic cancer. In a particular embodiment, the PRR detected is TLR7.

The present description is further illustrated by the following examples, which should not be construed as limiting in any way. The contents of all cited references (including literature references, issued patents, and published patent applications as cited throughout this application) are hereby expressly incorporated by reference.

### EXAMPLE

### Animals

Male C57BL/6 (H-2 Kb) mice and mice deficient in TLR7 (B6.129S1-Tlr7^{tmlFlv}/J), NF-κB (B6.129P-Nfkb^{tmlBal}/J), CD4⁺ T cells (B6.129S2-Cd4^{tmlMak}/J), CD8⁺ T cells (B6.129S2-Cd8^{tmlMas}/J), and B cells (B6.129S2-Ighm^{tmlCgn}/J) were purchased from Jackson Labs (Bar Harbor, Me.). p48Cre;Kras^{G12D} mice which develop pancreatic neoplasia endogenously by expressing a single mutant Kras allele in progenitor cells of the pancreas (gift of Dafna Bar-Sagi, New York University), were generated by crossing LSL-Kras^{G12D} mice with p48Cre mice, which express Cre recombinase from a pancreatic progenitor-specific promoter (Tuveson et al., 2004). Animals were housed in a clean vivarium and fed standard mouse chow. All animal procedures were approved by the New York University School of Medicine Institutional Animal Care and Use Committee.

### In Vivo Experiments

In vivo TLR7 ligation was accomplished by thrice weekly i.p administration of selective TLR7 agonists (ssRNA40, E. coli RNA, or Adenine analogue, all 100 μg/kg; Invivogen, San Diego, Calif.). TLR7 blockade was accomplished using an oligonucleotide inhibitor of TLR7 (Guiducci et al., 2010) (IRS661, 100 μg, 3× per week; Dynavax, Berkeley, Calif.). Bone marrow chimeric animals were created by irradiating mice (100 Gy) followed by i.v. bone marrow transfer (1×10⁷ cells) from non-irradiated donors as described (Bedrosian et al., 2011) Chimeric mice were employed in experiments 7 weeks later. In selected experiments, to accelerate carcinogenesis in p48Cre;Kras^{G12D} mice, two doses of caerulein (50 μg/kg) were administered over 48 hours as described (Carriere et al., 2009). MAP Kinase signaling blockade was accomplished using the MKK inhibitor PD98059 (2.5 mg/kg/day), the p38 inhibitor SB203580 (30 mg/kg/day), or the JNK inhibitor SP600125 (50 mg/kg/day; all Invivogen). Selective NF-κB signaling blockade was accomplished using either the NEMO binding domain inhibitor (1 mg/kg/day, EMD4Biosciences, Gibbstown, N.J.) or the IkB inhibitor Bay11-7082 (1.25 mg/kg/day; Invivogen). To deplete CD4⁺ T cells or CD8⁺ T cells, respectively, GK1.5, or 53-6.7 were employed (both 150 μg/3× weekly; Monoclonal Antibody Core Facility, Sloan-Kettering Institute, New York, N.Y.) as described (Miller et al., 2002). To study pancreatic inflammation in absence of mutated oncogenic Kras, chronic pancreatitis was induced in WT mice using a regimen of seven hourly i.p. injections of caerulein (50 μg/kg; Sigma-Aldrich) thrice weekly for 3-6 weeks. Alternatively, twice daily administration of L-arginine (40 mg/kg; Sigma-Aldrich, Saint Louis, Mo.) for one week was used to induce pancreatic injury as described (Bedrosian et al., 2011).

### Cellular Isolation and Analysis

To isolate mononuclear cells from the pancreas or spleen, organs were harvested via post-mortem laparotomy and processed using both mechanical and enzymatic digestion with Collagenase IV (Sigma-Aldrich) as described (Bedrosian et al., 2011; Connolly et al., 2009). Cell surface marker analysis was performed by flow cytometry using the FACS Calibur (Beckman Coulter; Brea, Calif.) after incubating 5×10⁵ cells with 1 μg of anti-FcγRIII/II antibody (2.4G2, Fc block; Monoclonal Antibody Core Facility, Sloan-Kettering Institute) and then labeling with 1 μg of FITC, PE, PerCP, or APC-conjugated antibodies directed against B220 (RA3-6B2), CD3ε (17A2), CD4 (RM4-5) CD8α (53-6.7), CD11b (M1/70), CD11c (HL3), CD19 (1D3), CD45 (30-F11), CD133 (315-2C11), and Gr1 (RB6-8C5) (all eBiosciences, San Diego, Calif. or BioLegend, San Diego, Calif.). To determine TLR7 expression, cells were fixed and permiablized and stained using a mAb directed against TLR7 (IMG-581A, Imgenex, San Diego, Calif.). Dead cells were identified and excluded from analysis by staining with 7-amino-actinomycin D (7AAD).

### Western Blotting and TLR7 Agonist Quantification

Western blotting was performed as described (Connolly et al., 2011). Briefly, pancreata were homogenized in RIPA buffer with Complete Protease Inhibitor cocktail (Roche, Pleasanton, Calif.) and proteins were separated from larger fragments by centrifugation at 14000×g. Alternatively, human pancreatic duct fluid harvested at surgery from patients undergoing pancreatic resection was utilized. After determination of total protein by the Lowry protein assay, 10% polyacrylamide gels were equiloaded with samples, electrophoresed at 90 V, electrotransferred to PVDF membranes and probed with monoclonal antibodies to ERK1, pERK, Elk1, pElk, IkBα, pIkBα, NF-κB, pNF-κB, HMGB-1, S100A9, and β-actin (Santa Cruz Biotechnology, Santa Cruz, Calif.). Blots were developed by ECL (Thermo Scientific, Asheville, N.C.). Levels of TLR7 in pancreatic lysates and human pancreatic duct fluid were quantified on HEK-Blue cells (Invivogen) using fixed quantities of TLR7 ligand (ssRNA40) as controls (Guo et al., 2009).

### Histology, Immunohistochemistry, and Microscopy

For histological analysis, pancreatic specimens were fixed with 10% buffered formalin, dehydrated in ethanol, and then embedded with paraffin and stained with H&E or Gomori's Trichrome. In addition, immunohistochemistry (IHC) was performed using antibodies directed against B220 (BD Phaimigen, Franklin Lakes, N.J.), CD3 (Invitrogen, Carlsbad, Calif.), p16 (Abcam, Cambridge, Mass.), p53 (Novocastra, Newcastle Upon Tyne, United Kingdom), CK19 (University of Iowa Developmental Studies Hybridoma Bank, Des Moines, Iowa), CD45R (BD Biosciences), α-SMA (Novus Biologicals, Littleton, Colo.), insulin (Abeam, Cambridge, Mass.), myeloperoxidase (Lifespan Biosciences, Seattle, Wash.), Ki67 (Thermo Fischer Scientific, Freemont, Calif.) and TLR7 (Imgenex). Photographs were taken with a Leica DM2M microscope (Leica Microsystems, Bannockburn, Ill.) and an Optronics Digital Camera (Optronics, Goleta, Calif.). Quantifications were performed by examining 10 high powered fields (HPF) per pancreas as described (Bedrosian et al., 2011). Grades of PanIN lesions were determined as described (Hruban et al Am J Surg Pathol 25, 579, 2001).

### Human Specimens

De-identified paraffin embedded tissue from 5 human resected acute pancreatitis specimens and 19 pancreatic cancer specimens were analyzed for TLR7 expression using immunohistochemistry. Human pancreatic duct fluid harvested at surgery from patients undergoing surgical resection was treated with Complete Protease Inhibitor cocktail (1:2 ratio) and stored frozen. All human tissues were collected using an IRB-approved protocol.

### Statistical Analysis

Data is presented as mean+/−standard error of mean. Statistical significance was determined by the Student's t test and or log-rank test using GraphPad Prism 5 (GraphPad Software, La Jolla, Calif.). p-values <0.05 was considered significant.

### Elevated Expression of TLR7 in Pancreatic Cancer

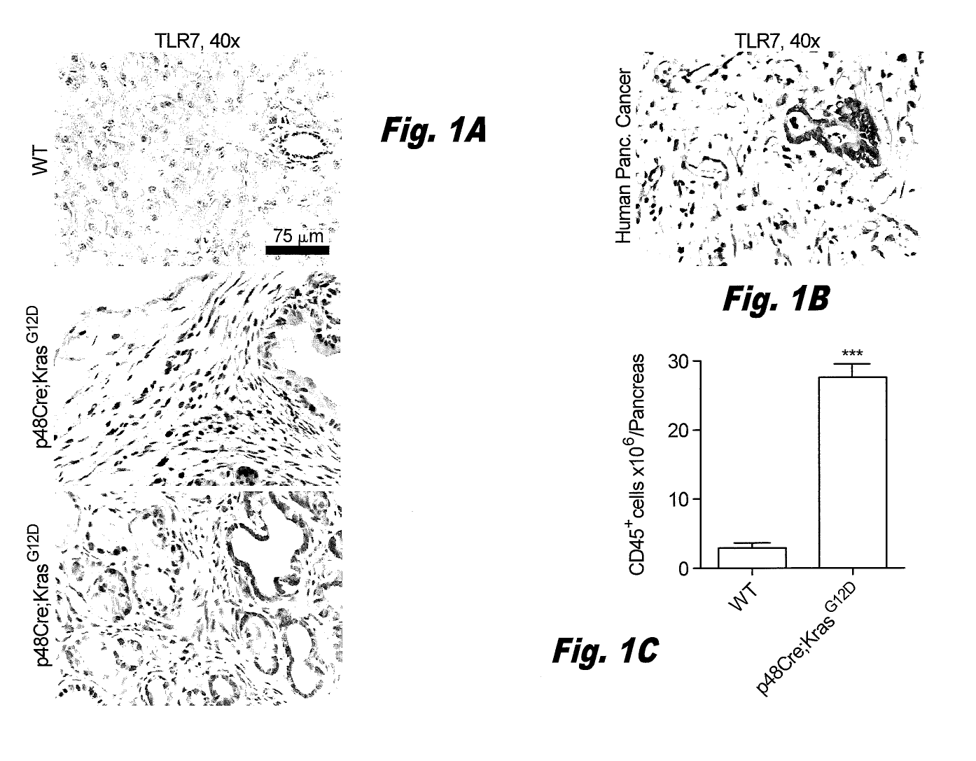

To determine the relevance of TLR7 to pancreatic cancer, using IHC, the inventors tested TLR7 expression in the normal pancreas, in murine models pancreatic cancer, and in human pancreatic adenocarcinoma. There was sparse expression of TLR7 in the normal mouse pancreas. Conversely, in pancreata of p48Cre; Kras^{G12D} mice both inflammatory stromal cells as well as neoplastic and dysplastic ductal epithelial cells robustly expressed TLR7 (FIG. 1A). Similarly, human pancreatic cancer specimens exhibited high expression of TLR7 in both neoplasitc epithelial cells and inflammatory cells within the stromal compartment (FIG. 1B).

To investigate which particular parenchymal and stromal cellular subset gained TLR7 expression in pancreatic cancer, single cell suspensions were isolated from normal and tumorous mouse pancreata and analyzed for TLR7 expression in CD45⁺ and CD45⁻ cellular subsets. There was a 10-fold increase in the absolute number of CD45⁺ leukocytes in the pancreata of 6 months old p48Cre; Kras^{G12D} mice as anticipated (FIG. 1C) (Beatty et al., 2011). Moreover, whereas leukocyte subsets harvested from normal pancreata expressed almost no TLR7, within the pancreas cancer tumor stroma, roughly 50% of B cells, 65% of T cells, 40% of neutrophils, and nearly all macrophages and dendritic cells expressed TLR (FIG. 1D). To determine whether cancerous pancreatic epithelial cells or endothelial cells also gain TLR7 expression, the inventors gated separately on CD34⁻CD45⁻ cells and co-stained for CD133 (ductal epithelial cell marker) and CD146 (endothelial cell marker). Consistent with our immunohistochemical observations, roughly 50% of Kras-transformed epithelial cells gained TLR7 expression (FIG. 1E). Conversely, only a small fraction of endothelial cells expressed TLR7 in both the normal and neoplastic pancreas (FIG. 1E).

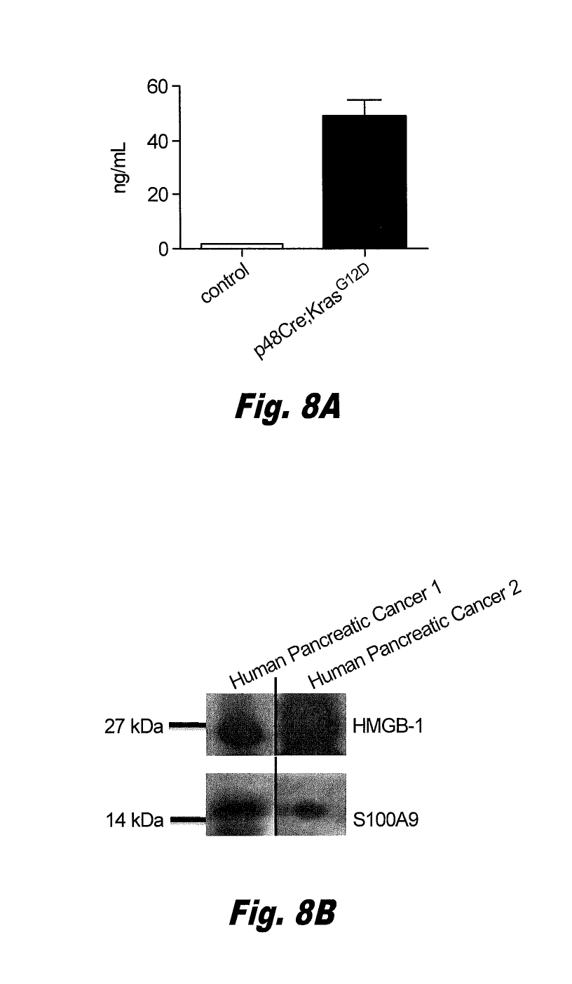

To further investigate the potential significance of TLR7 in pancreatic carcinoma, the inventors examined the prevalence of TLR7 ligands within the pancreatic cancer microenvironment. Specifically, the inventors tested both pancreatic tissue levels of TLR7 ligands in WT and p48Cre;Kras^{G12D} mice and pancreatic duct fluid levels of selected DAMPs in human pancreatic andeocarcinoma. Pancreatic lysates from p48Cre;Kras^{G12D} mice exhibited high levels of activating TLR7 agonists compared with WT pancreata when tested on TLR7 reporter cell lines (FIG. 8A). Similarly, pancreatic duct fluid from cancer patients contained high levels of HMGB-1 and S100A9 suggesting the presence of robust substrate for TLR7 activation within the pancreatic tumor microenvironment (FIG. 8B).

### TLR7 Ligation Accelerates Pancreatic Cancer Progression

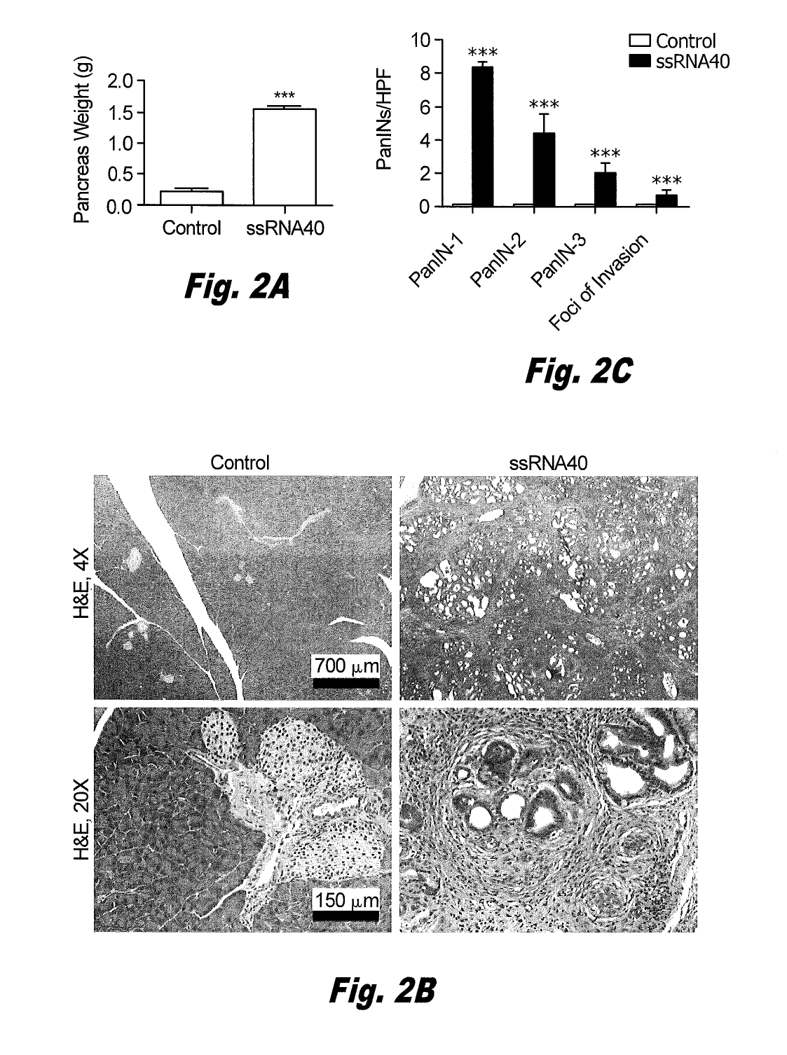

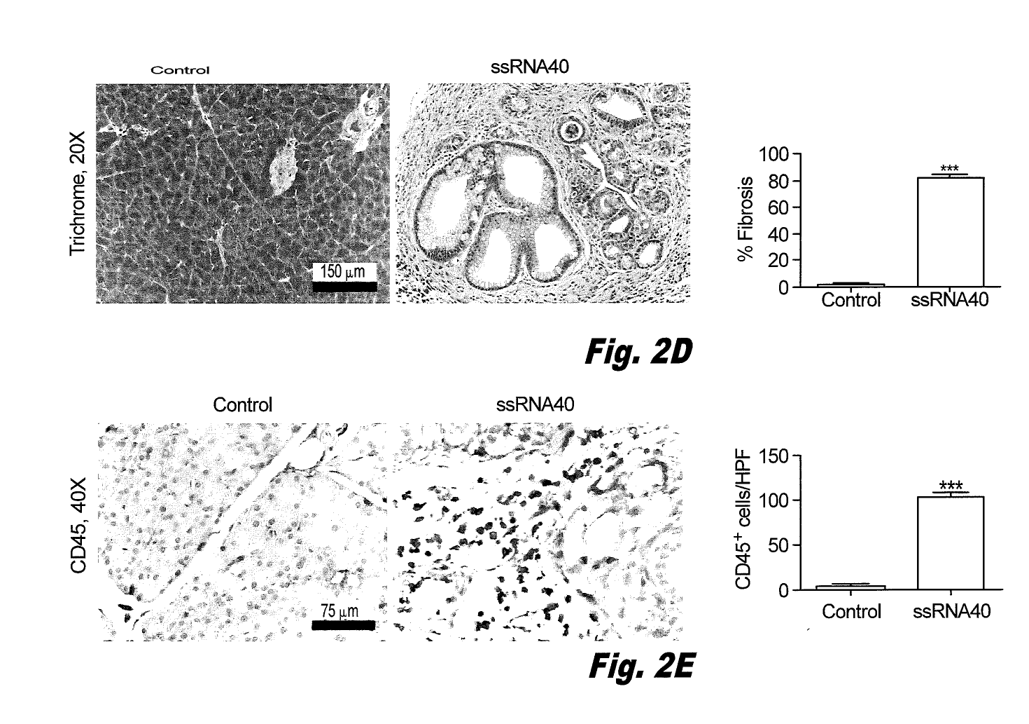

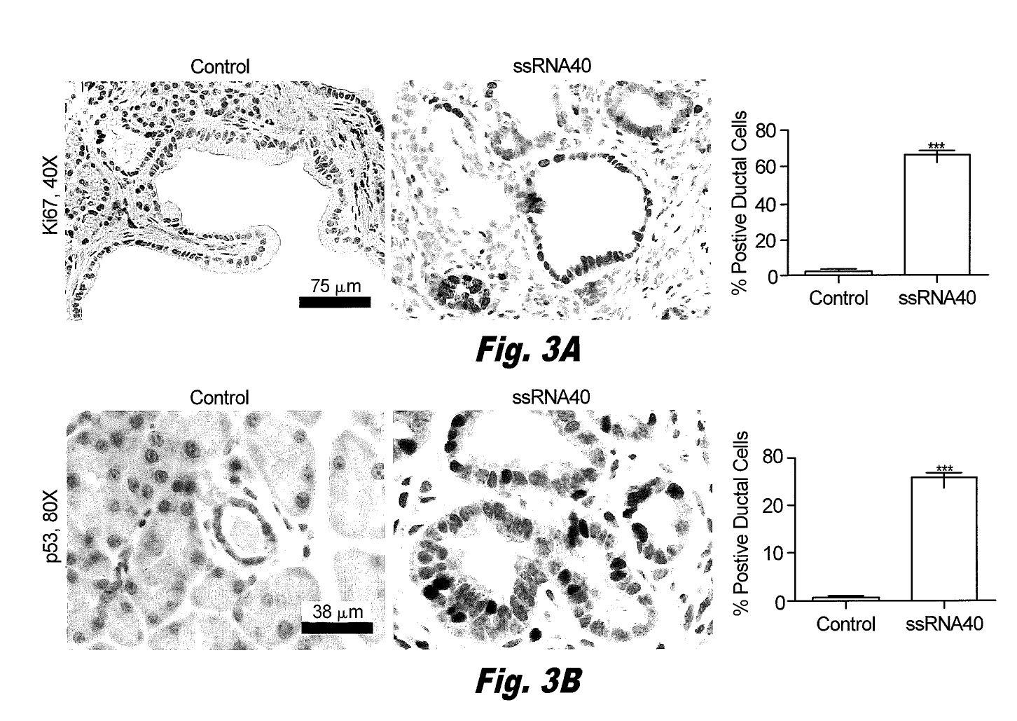

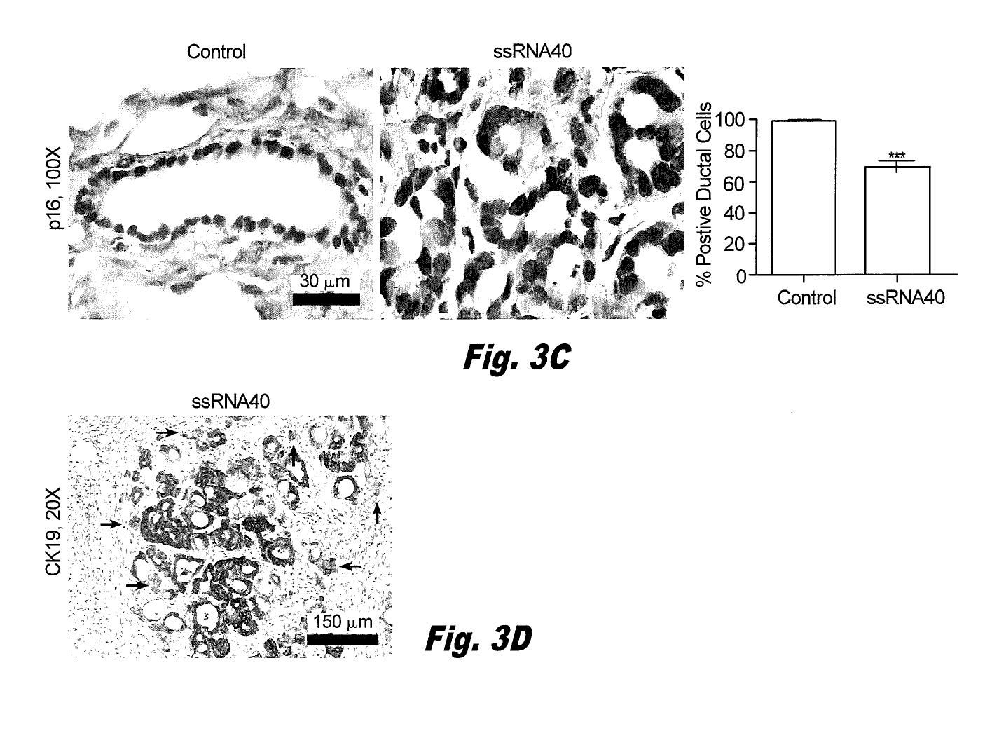

Since TLR7 is highly expressed in pancreatic cancer and TLR7 agonists are prevalent in the tumor microenvironment, the inventors postulated that TLR7 ligation accelerates tumor progression. To test this, the inventors treated six week old p48Cre;Kras^{G12D} mice with TLR7 ligand ssRNA40 or saline for three weeks before sacrifice. Pancreata harvested from TLR ligand treated animals were roughly three times larger than those of saline treated mice (FIG. 2A, B). Moreover, histologic analysis revealed that TLR7 ligation caused markedly advanced neoplasia and stromal desmoplasia. In particular, aged-matched saline-treated p48Cre;Kras^{G12D} mice had mostly normal pancreatic architecture with few scattered early PanINs and absent desmoplasia. Conversely, mice exposed to exogenous TLR7 ligand exhibited complete effacement of their pancreatic acini with diffuse PanIN-I-III lesions as well as foci of invasive carcinoma embedded in a dense fibro-inflammatory stroma (FIG. 2C-F). Moreover, epithelial cells from mice treated with TLR7 ligand exhibited markedly high proliferation rates, loss of p16 expression, as well as expression of mutated p53 (FIG. 3A-C), all of which are consistent with advanced pancreatic oncogenesis and imply that TLR7 ligation is sufficient to induce somatic mutations in at-risk pancreata. CK19 staining confirmed the presence of invasive cancer in ssRNA40 treated animals (FIG. 3D). Taken together, these data suggest that ligation of TLR7 is powerfully pro-tumorigenic within the pancreas and exerts effects on both stroma and transformed ductal cells.

### TLR7 Inhibition is Protective Against Pancreatic Cancer

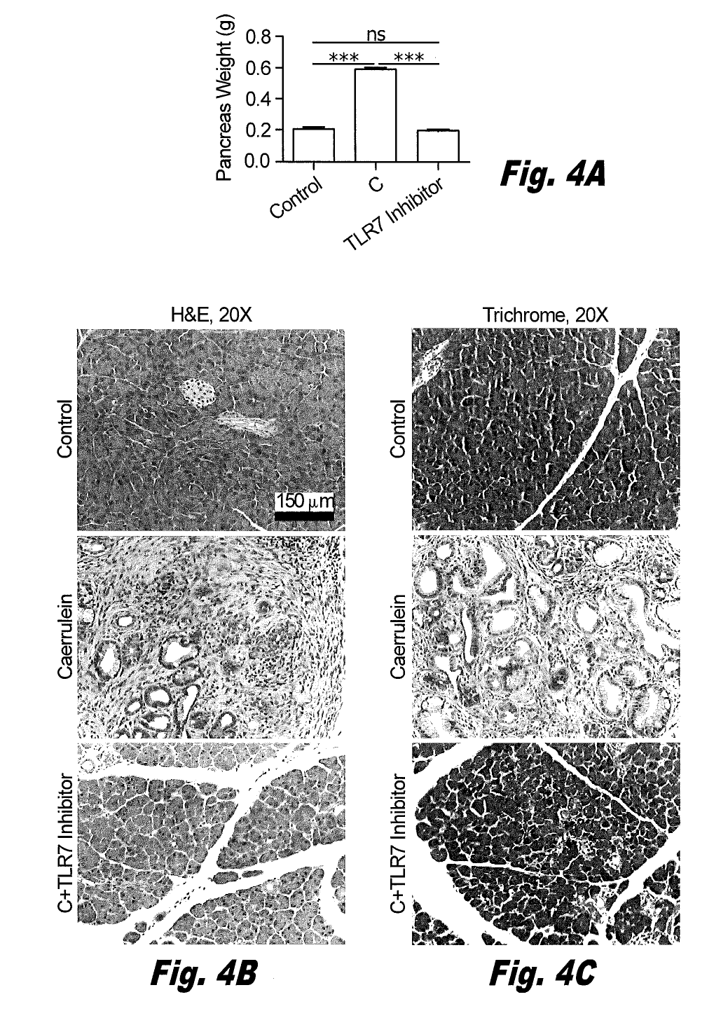

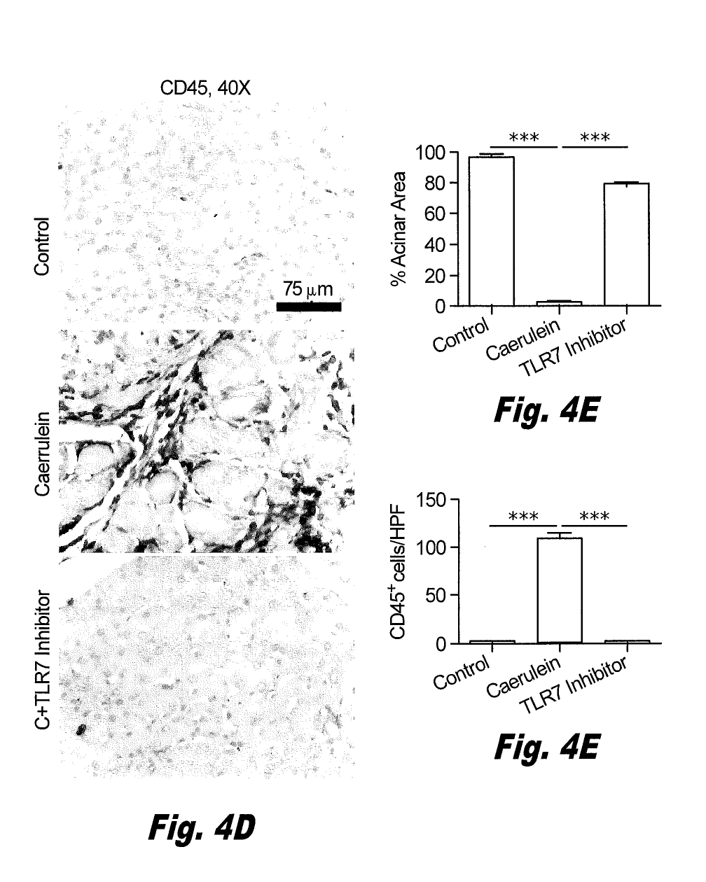

To determine whether TLR7 is essential for accelerated pancreatic carcinogenesis, the inventors treated cohorts of mice with caerulein for 2 days to accelerate cancer progression (Carriere et al., 2009). In parallel, selected mice were additionally treated with an oligonucliotide inhibitor of TLR7. Animals treated with caerulein developed invasive pancreatic adenocarcinoma within an extensive bed of fibro-inflammatory stroma as expected (FIG. 4A, B) (Carriere et al., 2009). Conversely, TLR7 blockade completely prevented malignant progression or stromal advancement. In particular, the effects of caerulein treatment on p48Cre;Kras^{G12D} mice in the context of TLR7 inhibition were entirely limited to mild pancreatic edema (FIG. 4B). These data imply that ligation of TLR7 is required for progression of pancreatic neoplasia and, conversely, blockade of TLR7 holds considerable promise in the treatment of pancreatic cancer.

### Ligation of TLR7 Exacerbates Pancreatic Inflammation and Fibrosis

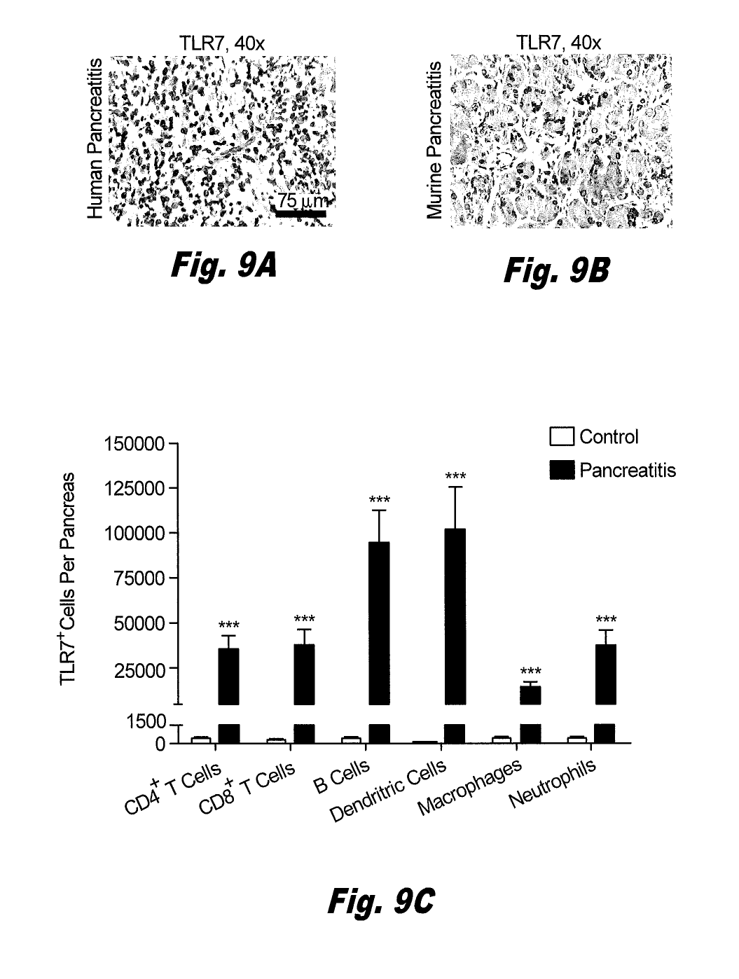

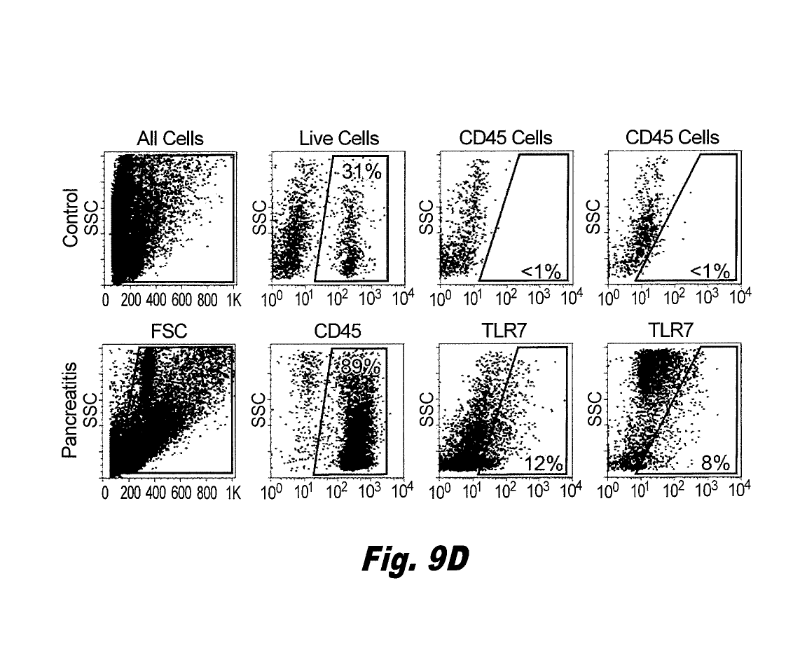

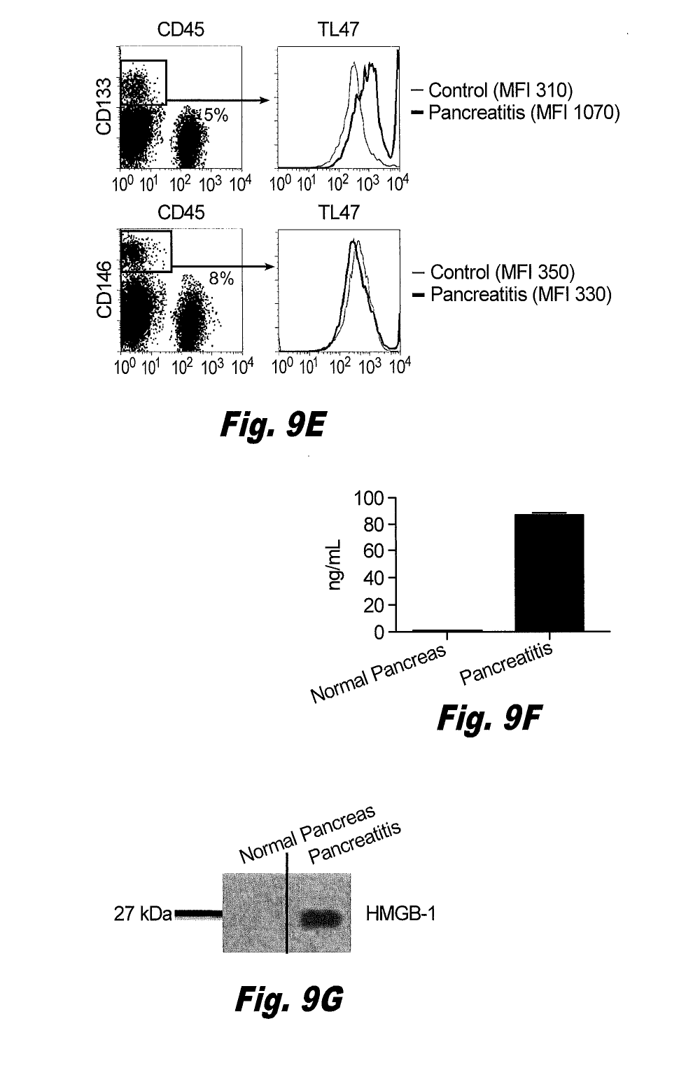

Since expansion of the fibro-inflammatory pancreatic tumor stroma has recently been found to be critical to pancreatic cancer progression (Kleeff et al., 1998) and the inventors found that TLR ligation induces vigorous stromal proliferation in mice harboring oncogenic Kras mutations in their pancreatic progenitor cells (FIGS. 2-4), the inventors postulated that TLR7 ligation exacerbates pancreatic carcinoma by directly inducing expansion of the fibro-inflammatory stroma. To examine this, the inventors first tested whether expression of TLR7 is altered in the pancreas during fibro-inflammatory disease, outside the context of oncogenic Kras mutation. The inventors found that in human chronic pancreatitis specimens as the inventorsll as in models of chronic pancreatitis in mice, a large fraction of infiltrating pancreas leukocytes stained positively for TLR7 (FIG. 9A, B). To examine early changes in the pancreatic leukocyte expression of TLR7 occurring during inflammation, the inventors employed a 24 h model of acute pancreatitis. Using flow cytometry, the inventors discovered a robust recruitment of TLR7 expressing CD4⁺ and CD8⁺ T cells, B cells, neutrophils, macrophages, and dendritic cells acute pancreatitis (FIG. 9C, D). Overall, roughly 10-15% of infiltrating CD45⁺ leukocytes were TLR7⁺ compared to virtually no expression in control pancreata. In addition, similar to pancreatic cancer, CD45⁻ parenchymal cells, including CD34⁻CD45⁻CD133⁺ ductal epithelial cells, also increased expression of TLR7 in the context benign pancreatic inflammation. Conversely, CD34⁻CD45⁻CD146⁺ endothelial cells maintained a constant level of TLR7 expression (FIG. 9E). The inventors also found high levels of TLR7 agonists in the pancreatic duct fluid of patients with pancreatitis and markedly elevated tissue levels of HMGB-1 in pancreata of WT mice experiencing pancreatitis (FIG. 9F, G). Taken together, these data suggest that intra-pancreatic inflammation is sufficient to increase TLR7 expression and levels of TLR agonists in the pancreas.

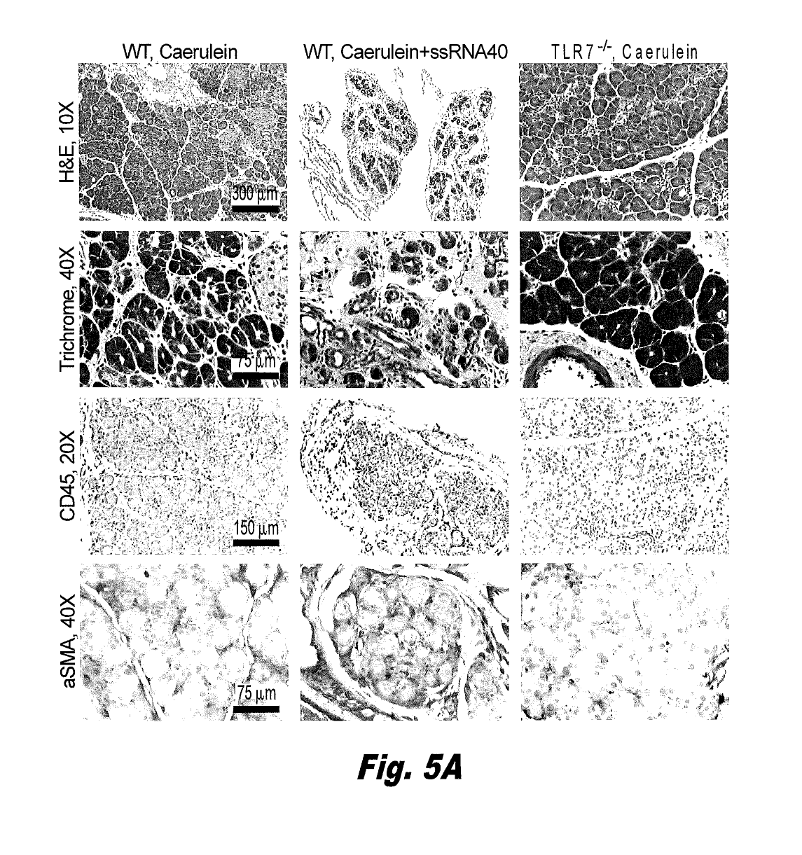

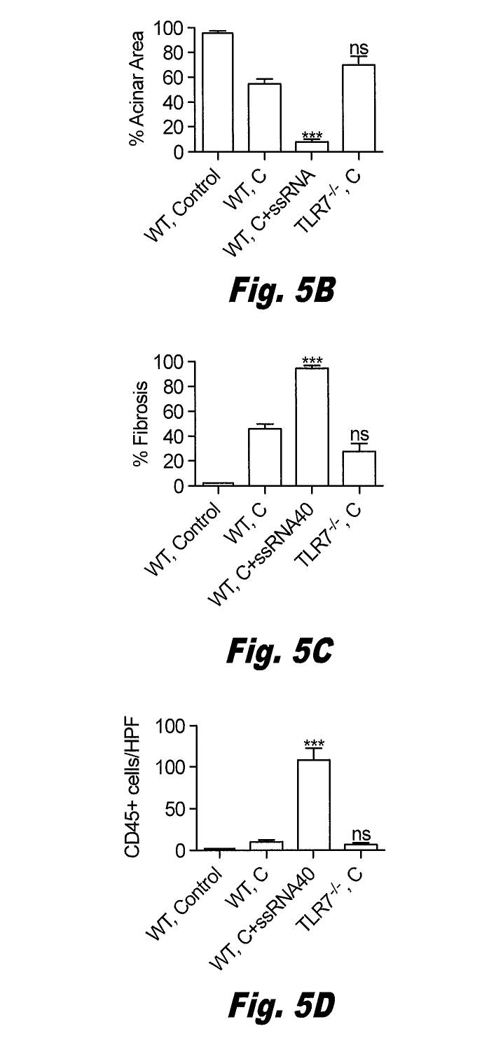

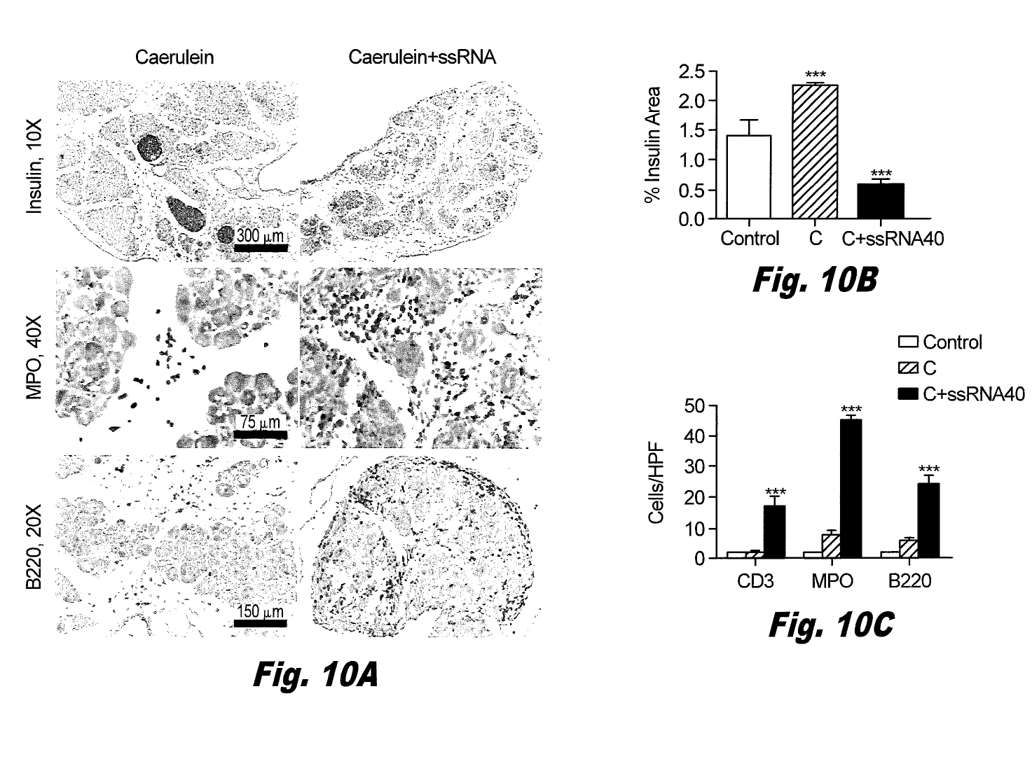

To directly test the effects of TLR7 ligation on fibro-inflammatory stromal expansion in the absence of mutated oncogenic Kras, the inventors induced chronic pancreatitis alone or supplemented with TLR7 ligand ssRNA40 in WT mice. Ligation of TLR7 resulted in markedly increased intra-pancreatic inflammation, fibrosis, as well as exocrine and endocrine organ destruction (FIG. 5A-D, FIG. 10A-C). The inventors also found high peri-acinar α-SMA expression after TLR7 ligation, implying activation of pancreatic stellate cells, which is a central cellular element responsible for stromal expansion in pancreatic carcinoma (FIG. 5A) (Omary et al., 2007).

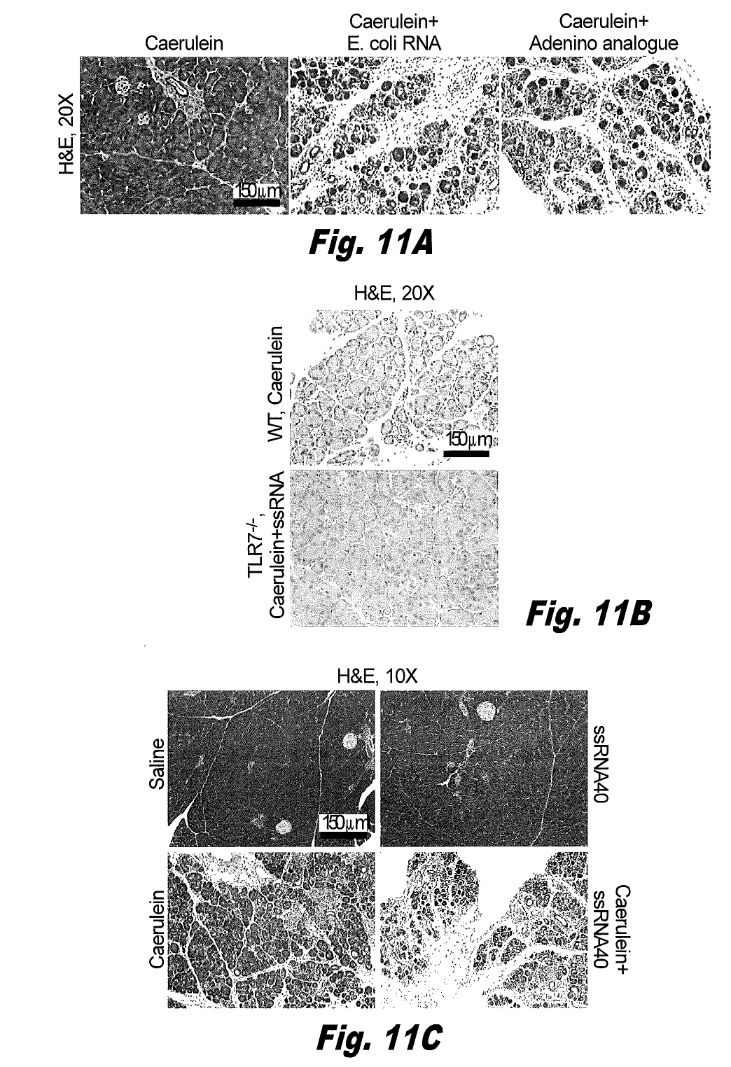

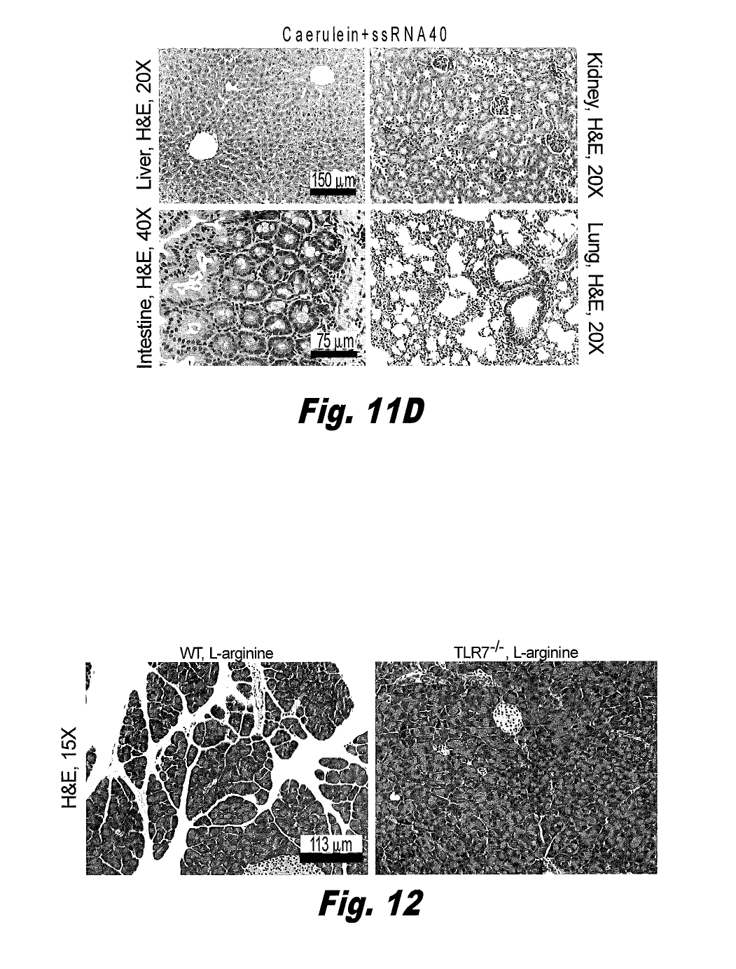

To investigate whether fibro-inflammatory effects were specific to ssRNA40 or generalizable to activating TLR7 ligands, the inventors tested other well characterized TLR7 ligands including E. coli ssRNA, and Adenine analog and found similar effects (FIG. 11A). To further test the specificity of the stromal expansive effects to TLR7 activation, the inventors administered ssRNA40 to TLR7^{-/-} mice. However, as anticipated, the inventors found no exacerbation of pancreatitis confirming the specificity of the fibro-inflammatory effects to TLR7 ligation (FIG. 11B). Furthermore, TLR7 agonists had no effects on expansion of pancreatic stroma in absence of a baseline level of inflammation (FIG. 11C). That is, administration of ssRNA40 to mice not experiencing pancreatitis had did not induce any intra-pancreatic stromal expansion, suggesting that the effects of TLR7 on stromal expansion requires synergy with other inflammatory elements (FIG. 11C). Furthermore, to determine whether fibro-inflammatory effects of TLR7 ligation were specific to the pancreas, the inventors examined liver, kidney, lung and intestine from WT mice treated with caerulein and ssRNA40 (FIG. 11D). The inventors found no evidence of extra-pancreatic effects on histological analysis suggesting that TLR7 ligation confers stromal expansive effects exclusively within the diseased pancreas.

### TLR7 Signaling is Required for Pancreatic Fibro-Inflammation

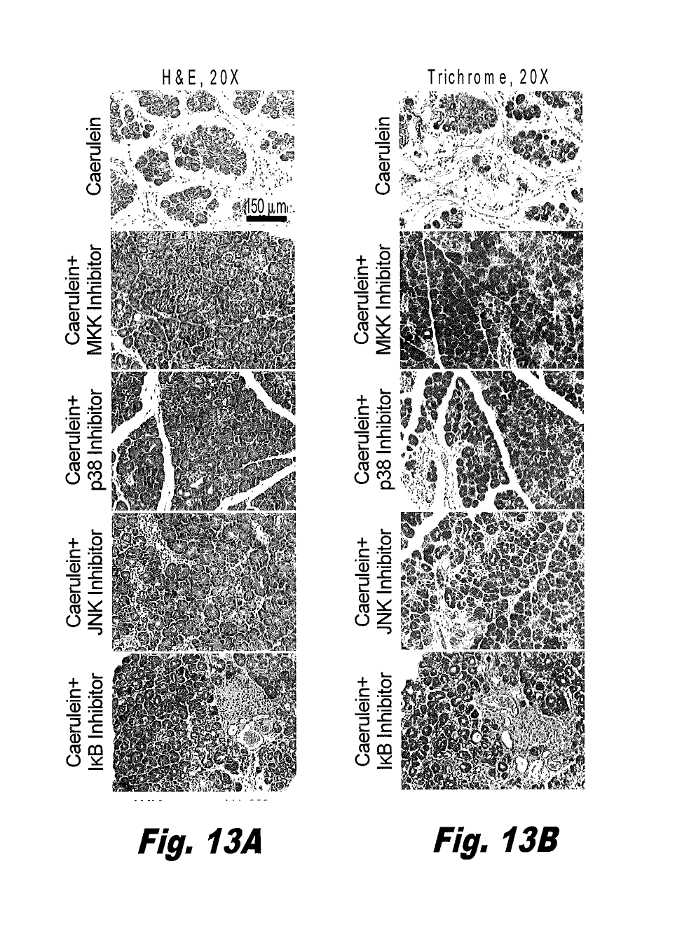

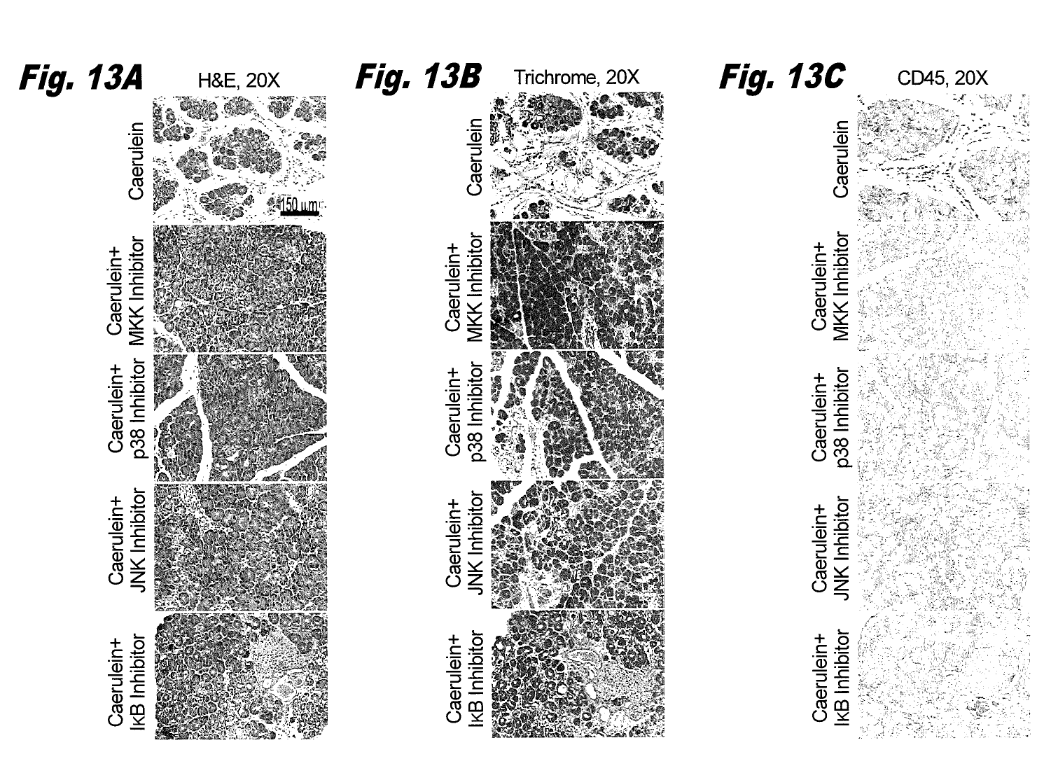

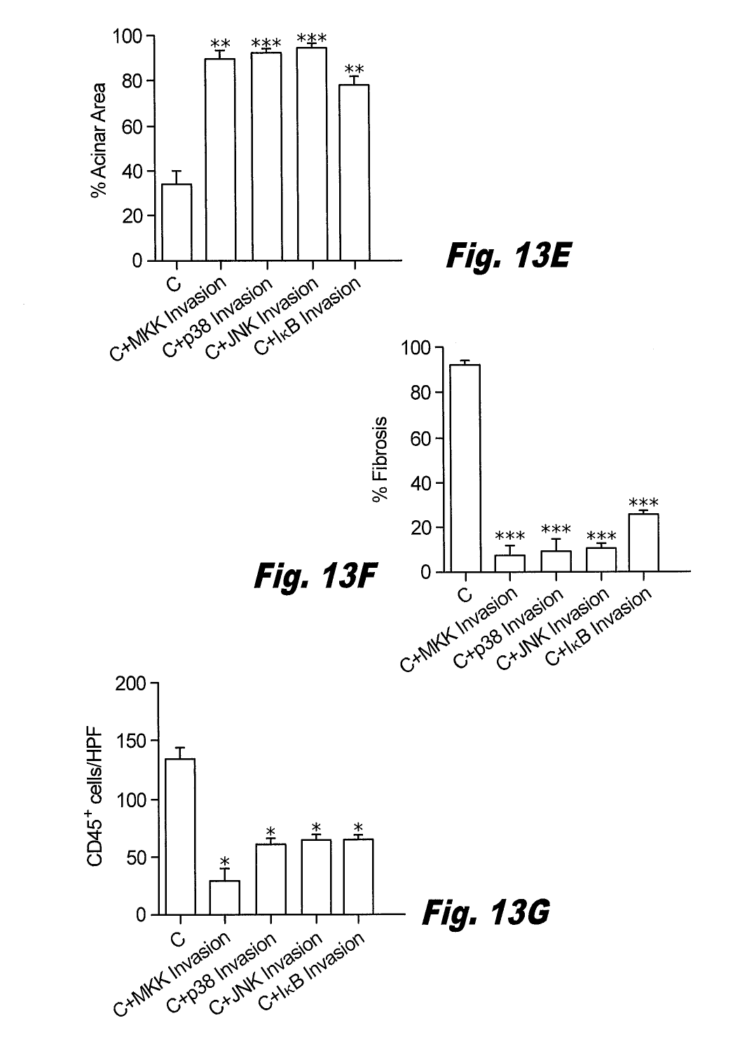

The inventors showed that targeted ligation of TLR7 is sufficient to exacerbate pancreatic inflammation and fibrosis. To determine whether signaling via TLR7 is necessary for pancreatic fibro-inflammation, the inventors treated TLR7^{-/-} mice with caerulein or L-Arginine. In both instances, TLR7^{-/-} mice were protected from pancreatitis (FIG. 5A-D, FIG. 12A) suggesting that TLR7 is essential for pancreatic stromal advancement. Since signaling downstream of TLR7 occurs via both the NF-κB and MAP-kinase pathways, the inventors examined the relative requirement for each signaling pathway for generation of stromal inflammation by selectively blocking downstream elements. The inventors found that selective inhibition of MKK, p38, JNK, or IκB, each had similar protective effects against intra-pancreatic fibro-inflammation (FIG. 13A-C).

### Inflammatory Cell TLR7 Signaling in Required for Progression of Pancreatic Carcinoma

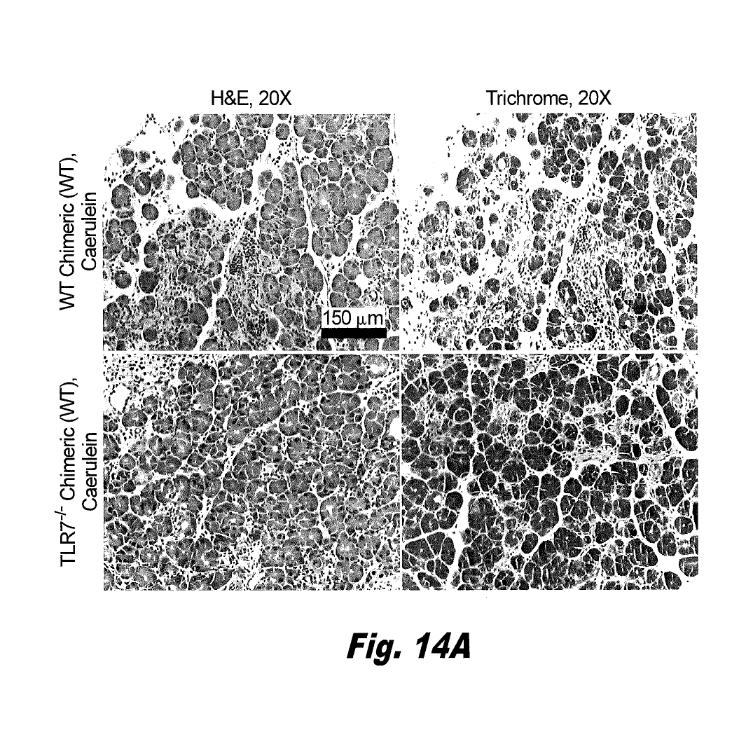

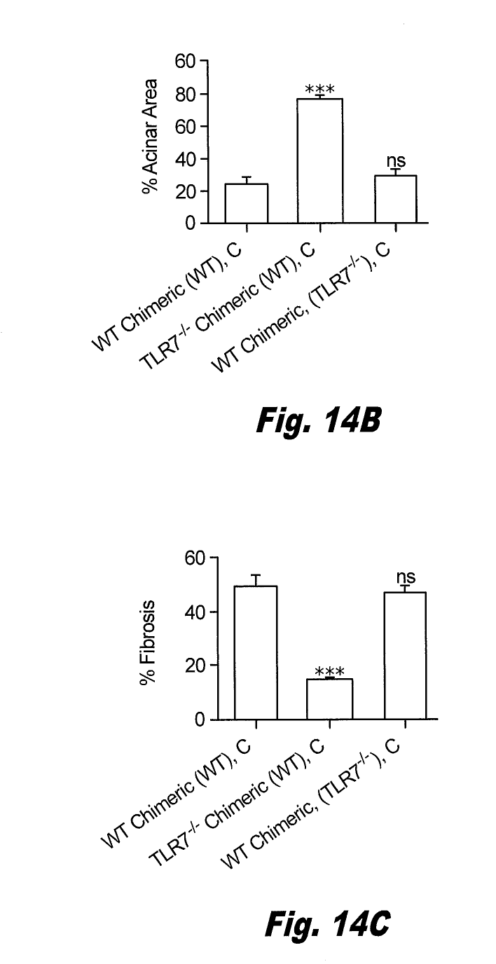

The inventors showed that both inflammatory and ductal cells gain expression of TLR7 in both benign and malignant pancreatic disease. To investigate specifically whether leukocyte or parenchymal cells expression of TLR7 in was the critical for exacerbated fibro-inflammation and neoplasitc progression, the inventors created both TLR7^{-/-} and WT bone marrow chimerics of WT mice so that immune cells in selected recipient mice were deficient in TLR7 whereas pancreatic parenchymal cells had intact TLR7 signaling. The inventors found that WT mice made chimeric with TLR7^{-/-} bone marrow were protected from benign pancreatic fibro-inflammatory disease (FIG. 14A-C). Conversely, TLR7^{-/-} made chimeric with WT bone marrow cells were not protected from pancreatic inflammation or fibrosis (FIG. 14B, C) implying that blockade of TLR signaling in pancreatic parenchymal cells is not essential for pancreatic fibro-inflammation to proceed.

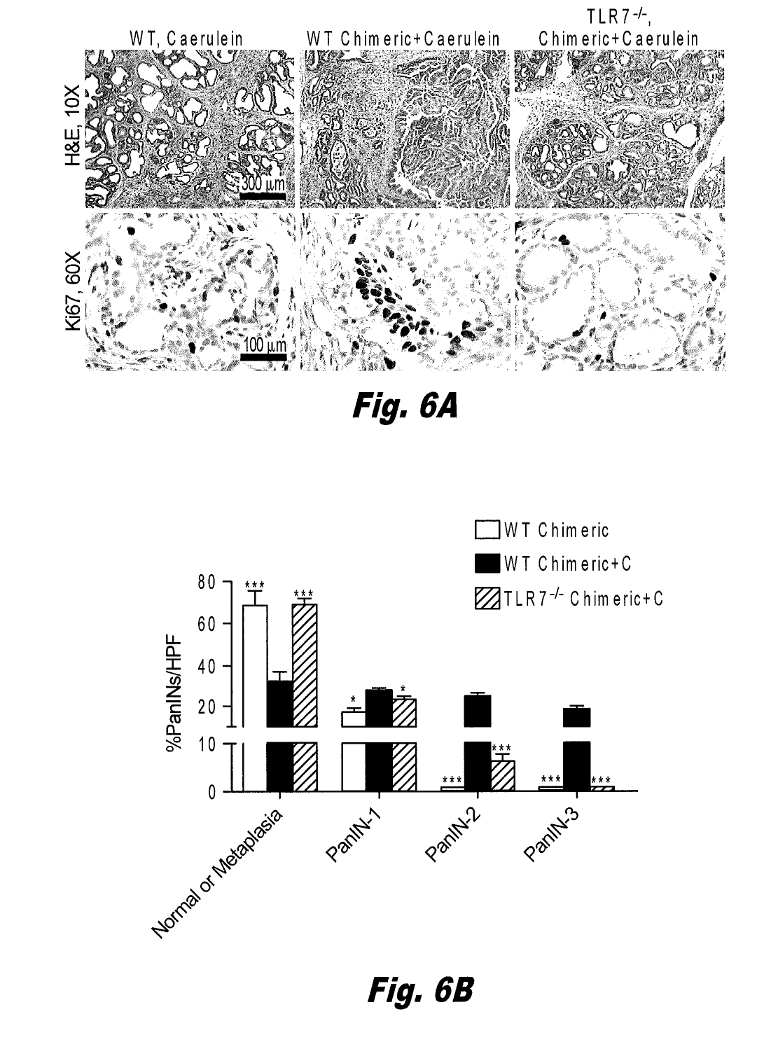

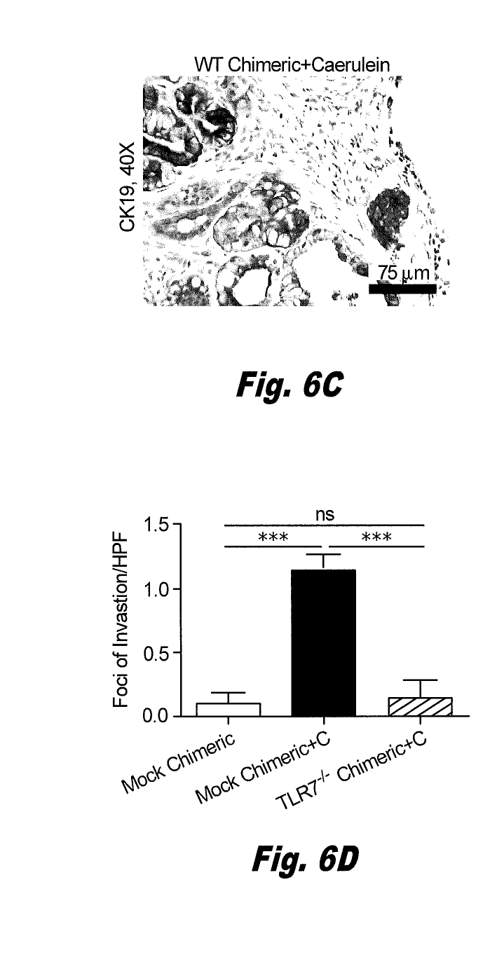

To test whether leukocyte expression of TLR7 is also necessary for pancreatic carcinoma to progress, the inventors irradiated 6 week old p48Cre;Kras^{G12D} mice and made them chimeric with bone marrow from TLR7^{-/-} mice or WT controls. Seven weeks later, at 13 weeks of life, selected cohorts were treated with 2 doses of saline or caerulein to accelerate carcinogenesis and mice were sacrificed 3 weeks later at 16 weeks of life. p48Cre;Kras^{G12D}-WT chimerics treated with saline had mostly metasplastic ducts with scattered early PanINs with low proliferation rates consistent with their age (FIG. 6A-C). Conversely, as expected, p48Cre;Kras^{G12D}-WT chimerics treated with caerulein developed advanced PanIN lesions with high proliferation rates and numerous foci of invasive cancer as exemplified by CK19 staining outside of ductal structures (FIG. 6A-D). Remarkably, however, p48Cre;Kras^{G12D}-TLR7^{-/-} chimerics were protected from accelerated neoplasia, indicating that TLR7 signaling within inflammatory cells is required for accelerated pancreatic carcinogenesis (FIG. 6A-D).

### TLR7 Ligation Exacerbates Pancreatic Disease Via Both MAP Kinase and NF-κB Pathways and Requires CD4⁺ T Cells

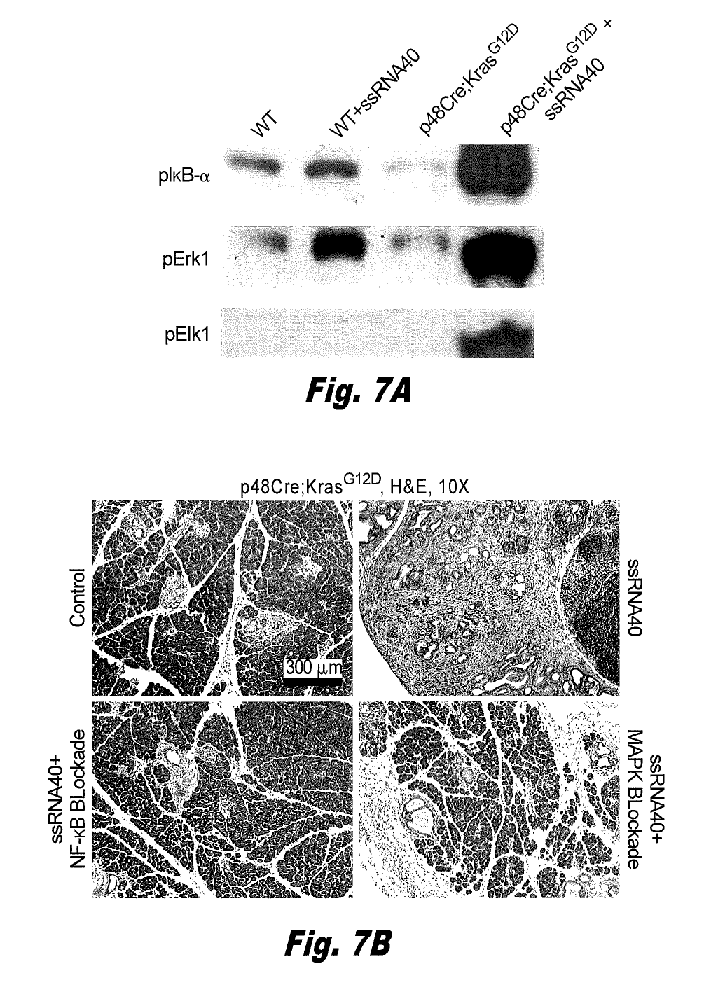

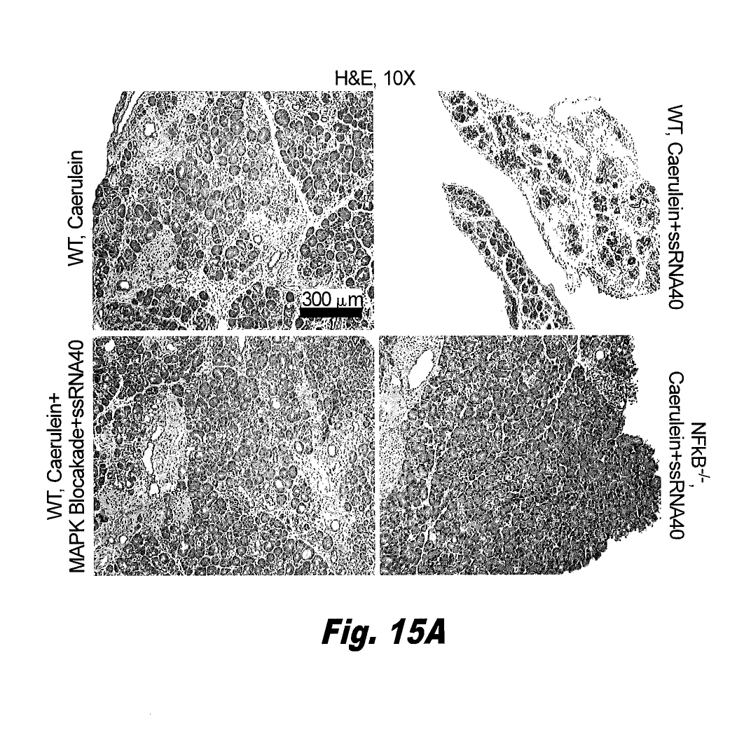

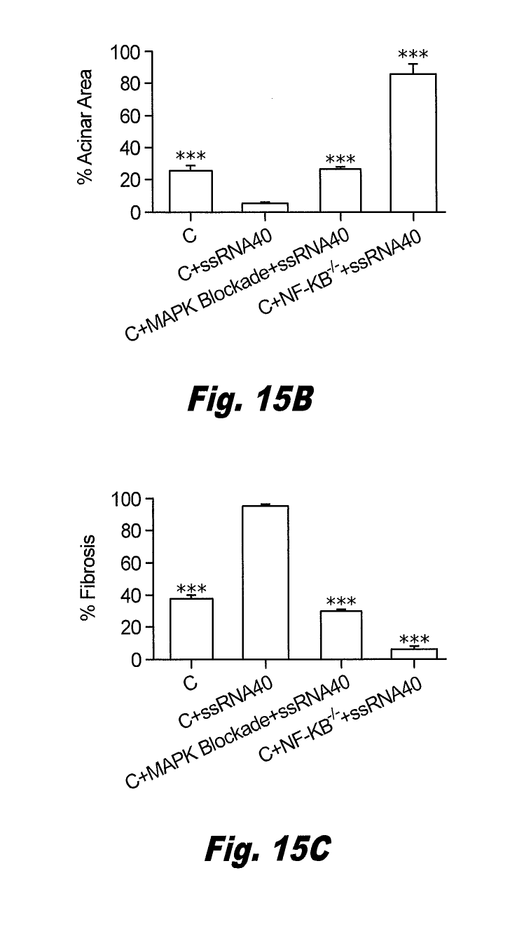

Since signaling via TLR7 can occur through both MAP Kinase and NF-κB pathways, the inventors postulated that TLR7 activation in p48Cre;Kras^{G12D} animals would induce elevated MAP Kinase and NF-κB signaling resulting in stromal expansion and accelerated tumorigenesis. Consistent with our hypothesis, pancreata from two-month old p48Cre;Kras^{G12D} mice treated with ssRNA40 expressed markedly elevated levels of pERK1, pElk, pIκB, and pNF-κB indicative of vigorous MAP Kinase and NF-κB signaling (FIG. 7a). To investigate the necessity for MAP Kinase and NF-κB intermediates in TLR7 mediated pancreatic carcinogenesis, the inventors selectively blocked each signaling pathway in p48Cre;Kras^{G12D} mice simultaneously treated with TLR7 agonists. Notably, MAP Kinase and NF-κB blockade each protected against the pro-tumorigenic effects of TLR7 ligation (FIG. 7b). Furthermore, consistent with our finding that TLR7 mediates its effect on the tumor stroma, blockade of either pathway mitigated TLR7 ligand effects on fibro-inflammatory pancreatic disease (FIG. 15).

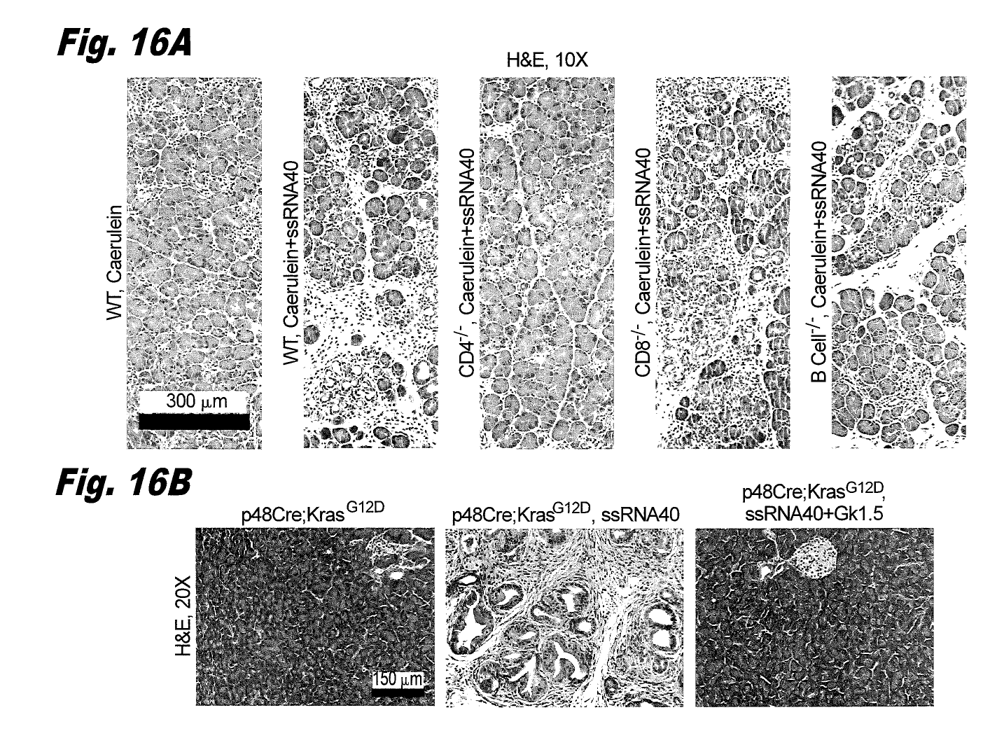

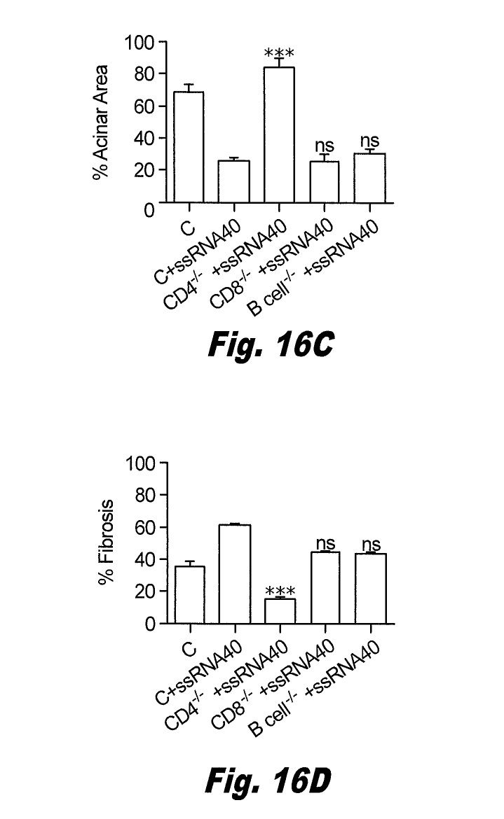

The inventors have recently shown that CD4⁺ T cells regulate pancreatic inflammation (Bedrosian et al., 2011). To investigate a possible link between TLR7 activation and the generation of CD4⁺ effector cells, the inventors first treated WT mice that had been depleted of CD4⁺ T cells with caerulein alone or caerulein+ssRNA40. CD4⁺ T cell depletion protected animals from the exacerbated fibro-inflammation associated with TLR7 activation (FIG. 16). Conversely, CD8⁺ T cell or B cell deficient mice were not protected (FIG. 16). Moreover, CD4⁺ T cells depletion protected p48Cre;Kras^{G12D} mice from the tumor promoting effects of TLR7 ligation (FIG. 7c). Taken together, these data show that CD4⁺ T cells are necessary for the accelerated stromal expansion and cancer progression induced by TLR7 signaling.

Beyond the overarching connections between TLR activation and progression of pancreatic cancer, this disclosure provides new mechanistic and empirical evidence in support of a sophisticated crosstalk between stromal and epithelial cells that drives carcinogenesis. Chimerization models provided herein demonstrate that TLR blockade within the tumor stroma is sufficient to halt cancer progression within the epithelial compartment. These findings are in consort with recent observations implicating the overriding role of stromal advancement on tumor progression. The complex stroma, which is comprised of activated fibroblasts and myofibroblasts, extracellular matrix, and diverse inflammatory cells, has been shown to produce an array of growth factors and inflammatory mediators including TGF-β, insulin-like growth factor 1, epidermal growth factor, and fibroblast growth factors, all of which provide nutrient support for neoplastic epithelial cells and drive tumor progression (Chu et al., 2007; Ide et al., 2006). Stromal advancement has been associated with significant permutations in the transcriptional program of transformed epithelial cells, affecting the expression and activity of matrix metalloproteinases, tissue inhibitors of metalloproteinases, VEGF, COX-2, HIF-1α, and others, all of which enhance cellular motility, induce neo-vascularization, or fortify resistance to hypoxia to collectively promote further tumor growth, invasion, and metastasis (Hwang et al., 2008b; Sato et al., 2004). Activation of the stroma via TLR7 ligation on inflammatory cells can result in additional somatic mutations in tumor suppressor genes. At the center of the stromal-epithelial crosstalk are CD4⁺ T cells, which play an integral role in both the inflammatory response to pathologic TLR activation. In particular, the cycle coupling stromal expansion and malignant transformation is arrested in the absence of CD4⁺ T cells suggesting that CD4⁺ T cells are the fundamental mediators between receptor ligation, stromal advancement, and tumor progression.

Additional PRRs such as TLR4 and TLR9 have been implicated in pancreatic inflammation (Hoque et al., 2011; Sharif et al., 2009; Zeng et al., 2008). However, their role in pancreatic neoplasia has not been previously explored. The Examples presented herein demonstrate the importance of TLR signaling and TLR-mediated inflammation in pancreatic cancer; therefore, the example of TLR7 can be directly extended to include TLR4 and TLR9 as additional modulators of pancreatic inflammation leading to pancreatic cancer, as well as the CLR dectin-1. Thus, antagonists of TLR4, TLR9, and dectin-1 will treat and prevent pancreatic cancer and inflammation in a manner similar to antagonists of TLR7. Further, this disclosure provides methods, antagonists, and compositions directed toward treating the synergistic pro-tumorigenic effects resulting from ligation of multiple PRRs within the desmoplastic tumor stroma. Simultaneous inhibition of multiple pattern recognition receptors can have additional anti-cancer protective effects over inhibition of TLR7 alone.

The inventors have discovered that human pancreatic adenocarcinomas robustly upregulate expression of TLR7 in both epithelial and stromal compartments. Whereas normal pancreata do not express significant TLR7—or contain TLR7 agonists—this receptor is highly expressed in both transformed epithelial cells and stromal cells in pancreatic cancer and its cognate ligands were found at elevated levels within the pancreatic cancer microenvironment. Based on these data, engagement of TLR7 within the tumor stroma emerges as a fundamental requirement for stromal advancement and pancreatic cancer progression. Targeting TLR7 or its relevant ligands within the tumor microenvironment is an effective treatment for human pancreatic cancer.

TLR7 ligation vigorously accelerates pancreatic tumor progression and induces additional p53 and p16 mutations in Kras-transformed pancreata. Conversely, blockade of TLR7 protects completely against pancreatic cancer. TLR7 ligation acts to modulate pancreatic carcinogenesis by regulating stromal activation. Accordingly, TLR7^{-/-} bone marrow chimeric p48Cre;Kras^{G12D} mice are protected from neoplastic advancement. TLR7 modulation of pancreatic carcinogenesis relies on both NF-κB and MAP kinase signaling pathways and required effector CD4⁺ T cells. Based on these data, targeting TLR7 activation holds promise for the treatment of human pancreatic cancer.

The inventors have shown that PRR signaling is a principal modulator of the pancreatic tumor microenvironment that drives stromal advancement and epithelial mutagenesis. Hence, timely translation of these results to a Phase I clinical is indicated to improve the bleak treatment landscape for patients with pancreatic cancer.

1. A method of treating or preventing pancreatic cancer in a subject, comprising administering a therapeutically effective amount of an antagonist of at least one pattern recognition receptor chosen from TLR4, TLR7, TLR9, and dectin-1.

2. The method of claim 1, further comprising administering one or more additional anti-cancer treatments to said subject.

3. The method of claim 2, wherein the one or more additional anti-cancer treatments is selected from the group consisting of surgery, radiation therapy, chemotherapy, and biological therapy.

4. A method of treating or preventing pancreatic inflammation in a subject, comprising administering a therapeutically effective amount of an antagonist of at least one pattern recognition receptor chosen from TLR4, TLR7, TLR9, and dectin-1.

5. The method of claim 1 or 4 wherein said antagonist is selected from the group consisting of: peptides, polypeptides, proteins, antibodies, antisense oligonucleotides, ribozymes, small molecules, chemotherapeutic agents, and fragments, derivatives and analogs thereof.

6. The method of claim 1 or 4, wherein the antagonist is a TLR7 antagonist.

7. The method of claim 6, further comprising administration of at least one of a TLR4 antagonist, a TLR9 antagonist, or a dectin-1 antagonist.

8. The method of claim 6, further comprising administering one or more additional anti-inflammatory treatments to said subject.

9. The method of claim 6, wherein one of said one or more additional anti-inflammatory treatments is a non-steroidal anti-inflammatory drug (NSAIDs) or an anti-inflammatory steroid.

10. A pharmaceutical composition for use in treating or preventing pancreatic cancer in a subject, comprising an effective amount of an antagonist of at least one pattern recognition receptor chosen from TLR4, TLR7, TLR9, and dectin-1.

11. A pharmaceutical composition for use in treating or preventing pancreatic inflammation in a subject, comprising an effective amount of an antagonist of at least one pattern recognition receptor chosen from TLR4, TLR7, TLR9, and dectin-1.

12. The pharmaceutical composition of claim 10 or 11, wherein the antagonist is selected from the group consisting of: peptides, polypeptides, proteins, antibodies, antisense oligonucleotides, ribozymes, small molecules, chemotherapeutic agents, and fragments, derivatives and analogs thereof.

13. The pharmaceutical composition of claim 10 or 11, wherein the antagonist is a TLR7 antagonist.

14. The pharmaceutical composition of claim 13, further comprising at least one of a TLR4 antagonist, a TLR9 antagonist, or a dectin-1 antagonist.

15. The pharmaceutical composition of claim 13, further comprising one or more additional anti-inflammatory compounds.

16. The pharmaceutical composition of claim 13, further comprising a non-steroidal anti-inflammatory drug (NSAIDs) or an anti-inflammatory steroid.

17. A method of screening for an candidate agent for the treatment of pancreatic cancer, comprising contacting a pattern recognition receptor (PRR) with a test compound and detecting PRR activity in the presence of the test compound relative to PRR activity in the absence of the test compound, wherein a decrease in PRR activity in the presence of the test compound relative to PRR activity in the absence of the test compound indicates that the test compound is a candidate agent for the treatment of pancreatic cancer.

18. The method of claim 17, wherein the pattern recognition receptor is TLR4, TLR7, TLR9, or dectin-1.

19. The method of claim 18, wherein the activity detected is signaling activity.

20. A method of determining individual susceptibility to development of pancreatic cancer, comprising:
a. detecting the level of TLR7 in a sample of pancreatic tissue from said subject; and
b. comparing the level of TLR7 in said subject to the level of TLR7 in a sample of pancreatic tissue from a control subject;
where an increase in the level of TLR7 in the pancreatic tissue sample of said subject relative to the level of TLR7 in the pancreatic tissue sample of said control subject indicates that said subject has increased susceptibility to the development of pancreatic cancer.
